(19) United States
(12) Patent Application Publication
(10) Pub. No.: US 2024/0254157 A1
(43) Pub. Date: Aug. 1, 2024

(54) MACROLIDE COMPOUNDS

(71) Applicant: VANDERBILT UNIVERSITY, Nashville, TN (US)

(72) Inventors: Brian O. BACHMANN, Nashville, TN (US); Benjamin REISMAN, Nashville, TN (US); Michael SAVONA, Nashville, TN (US); Haley RAMSEY, Nashville, TN (US); Gary SULIKOWSKI, Nashville, TN (US)

(21) Appl. No.: 18/563,804

(22) PCT Filed: May 27, 2022
(86) PCT No.: PCT/US2022/031403

§ 371 Date: Nov. 22, 2023

Related U.S. Application Data
(60) Provisional application No. 63/193,959, filed on May 27, 2021.

(51) Int. Cl. C07H 17/08; A61P 35/00

(52) U.S. Cl. CPC C07H 17/08; A61P 35/00

(57) ABSTRACT

Disclosed herein are macrolide compounds with ATP synthase inhibitory activity. The macrolides may be used to treat cancer and other proliferative disorders. The macrolides may be used to treat leukemia, including acute myeloid leukemia. The macrolides may be used to treat cancer, including AML, in a patient that has developed multidrug resistance. In some embodiments, the macrolide is a derivative of ammocidin A.

### CROSS-REFERENCE TO RELATED APPLICATION

This application claims the benefit of U.S. Provisional Application 63/193,959, filed May 27, 2021, the contents of which are hereby incorporated in its entirety.

### ACKNOWLEDGEMENT OF GOVERNMENT SUPPORT

This invention was made with government support under grant number CA226833 awarded by the National Institutes of Health. The government has certain rights in the invention.

### FIELD OF THE INVENTION

The invention is directed to novel macrolide compounds having ATP synthase/ATPase inhibitory activity. The compounds are useful in a variety of therapeutic settings, including as anticancer agents, antibiotics, and immunosuppressants.

### BACKGROUND

Cancer is a disease that can be characterized by abnormal clonal cellular proliferation, poor cell differentiation, and infiltration into other tissues and organs, bone marrow, and peripheral blood. Acute myeloid leukemia (AML) is a malignant clonal disease originating from hematopoietic stem cell and progenitors which has a 5-year survival rate of less than 30%. AML is often treated with cytotoxic chemotherapy which may yield between 30-75% remissions, but relapses are common. The resistance of leukemia cells to standard cytotoxic chemotherapy drugs remains the main obstacle in the treatment of AML. Tumor drug resistance is mainly divided into primary drug resistance and acquired drug resistance. Primary drug resistance connotes a native lack of sensitivity to drug therapy prior to the use of antitumor drugs (eg. Cytotoxic chemotherapy and failure to affect non-cycling cells in the nonproliferative GO phase). Acquired resistance refers to the loss of sensitivity to chemotherapy over time due tumor-derived molecular resistance mechanisms which render previously effective drugs ineffective.

Macrolides comprise a structurally and pharmacologically diverse class of natural products, selectively addressing an equally diverse array of cellular targets, such as immunosuppressive signaling (FK-506, FKB12 calcineurin), splicing factors (SF3b, pladienolide), ribosomes (azithromycin, erythromycin), and ion channels (ivermectin, glutamate gated chloride channel). Moreover, variations within structural families can possess entirely different targeting properties, which has motivated significant activity towards generating modified macrolides, via both chemical synthesis and biosynthetic pathway engineering. Correspondingly, the impact of macrolides has been realized in both the clinic and as chemical biological tools for uncovering new insights into cell biology.

Apoptolidin A (FIG. 1) was originally discovered in a screen for selective inducers of apoptosis in E1A oncogene transformed cell lines and was isolated from a strain designated Nocardiopsis sp. FU40‡. Remarkably, apoptolidin was 1,000-fold more potent against transformed cells (10 nM) than untransformed lines (10 μM). Further evaluation of the compound against the NCI-60 collection revealed that activity was greatest (low nanomolar) in cell lines that do not exhibit the Warburg effect, instead relying on oxidative phosphorylation. A suite of indirect cellular and biochemical studies are consistent with the molecular target of apoptolidin A being the F_{O}F₁ ATP synthase. However, cell line differential selectivity and cellular response profiles of the apoptolidins differ in comparison to the validated F_{O} subunit inhibitor oligomycin, suggesting a different mode of action for this family. Recent studies have demonstrated that apoptolidin family macrolides bind to the Ft subcomplex of ATP synthase, at a distinct allosteric site compared to previously described ATP synthase inhibitors. The structurally related natural product, ammocidin A (FIG. 2), was isolated from Saccharothrix sp. AJ9571 in a screen against Ras oncogene transformed cell lines. Amycolatopsin (FIG. 3), another structurally related macrolide, was isolated from Amycolatopsis sp. MST-108494.

While the naturally occurring macrolides possess intriguing physiological properties, they are not necessarily ideal as drug substances due to limitations relating the solubility, bioavailability, stability, or selectively. There remains a need for ATP synthase inhibitors with increased potency, improved pharmacokinetic profiles, chemical stability, and/or reduced toxicity. There remains a need for improved therapeutics for the treatment of proliferative disorders, including cancer, especially leukemia. There remains a need for improved therapeutic agents for the treatment of AML and other cancers that do not loss efficacy over time. There remains a need for improved cancer therapeutics which do not activate or exacerbate multi-drug resistance, in cancer generally and leukemia especially. There remains a need for improved cancer therapeutics which are effective in cancers that have developed multidrug resistance.

### SUMMARY

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts the chemical structure of apoptolidin A.

FIG. 2 depicts the chemical structure of ammocidin A.

FIG. 3 depicts the chemical structure of amycolatopsin A.

FIG. 4a depicts ApoJ/K analysis and biochemical bypass. ApoJ/K is required for methoxymalonyl-ACP loading but not for biochemical bypass using starter unit surrogates.

FIG. 4b depicts starter unit surrogates synthesized and incubated with apoJK null strain.

FIG. 4c depicts HPLC/MS analysis of targeted deletion of apoJK and chemical complementation with natural loading units, advanced diketide, and starter unit analogs.

FIG. 5A depicts the structure of KK-32-011/VU936203A.

FIG. 5B depicts the structure of KK-32-011/VU936203B.

FIG. 5C depicts the structure of KK-32-012/VU936195A.

FIG. 5D depicts the structure of KK-32-012/VU936195B

FIG. 6 depicts viability in the MV-4-11 human leukemia cell line as a function of ammocidin derivatives in an MTT viability assay.

FIG. 7 depicts comparable efficacy in a MV-4-11 cell line derived xenograft model between ammocidin A (square data points), venetoclax+azacyctidine (triangle data points), and venetoclax+azacyctidine+ammocidin (star data points).

FIG. 8 depicts pharmacokinetic data in mice dosed at 0.25 mg/kg with ammocidin A and ammocidin derivatives. In the case of analogs VU936195B (195B) and VU936203B (203B), only free ammocidin A could be detected.

FIG. 9A depicts comparative data against AML cell line Molm-13 for venetoclax and ammocidin A over 72 hours.

FIG. 9B depicts comparative data against AML cell line MV-4-11 for venetoclax and ammocidin A over 72 hours.

FIG. 9C depicts comparative data against AML cell line OCI-AML-3 for venetoclax and ammocidin A over 72 hours.

FIG. 9D depicts comparative data against AML cell line KMS-12 for venetoclax and ammocidin A over 72 hours.

FIG. 10A depicts Molm-13 cell viability 48 hours post-exposure to combinations of ammocidin A and venetoclax. FIG. 10B depicts synergy scores for combinations of ammocidin A and venetoclax against Molm-13 cells.

FIG. 11A depicts MV-4-11 cell viability 48 hours post-exposure to combinations of ammocidin A and venetoclax. FIG. 11B depicts synergy scores for combinations of ammocidin A and venetoclax against MV-4-11 cells.

FIG. 12A depicts primary AML cells viability 48 hours post-exposure to combinations of ammocidin A and venetoclax. FIG. 12B depicts synergy scores for combinations of ammocidin A and venetoclax against patient derived primary AML cells.

FIG. 13A depicts KMS-12 cell viability 72 hours post-exposure to combinations of ammocidin A and venetoclax. FIG. 13B depicts synergy scores for combinations of ammocidin A and venetoclax against KMS-12 cells.

FIG. 14A depicts ammocidin A activity against three primary AML patient samples using viability GI50 data from Prism (GI50 from double log regression). 18-12-001=0.0302 μM (0.0312 μM); 17-03-006=0.0103 μM (0.0321 μM); 29-09-002=0.0158 μM (0.0217 μM). Data collected Jul. 8, 2021.

FIG. 14B depicts ammocidin A activity against two primary AML patient samples using viability GI50 data from Prism (GI50 from double log regression). 18-12-001=0.0284 M (0.07536 μM); 17-03-006=0.0095 μM (0.0308 μM). Data collected Mar. 8, 2022.

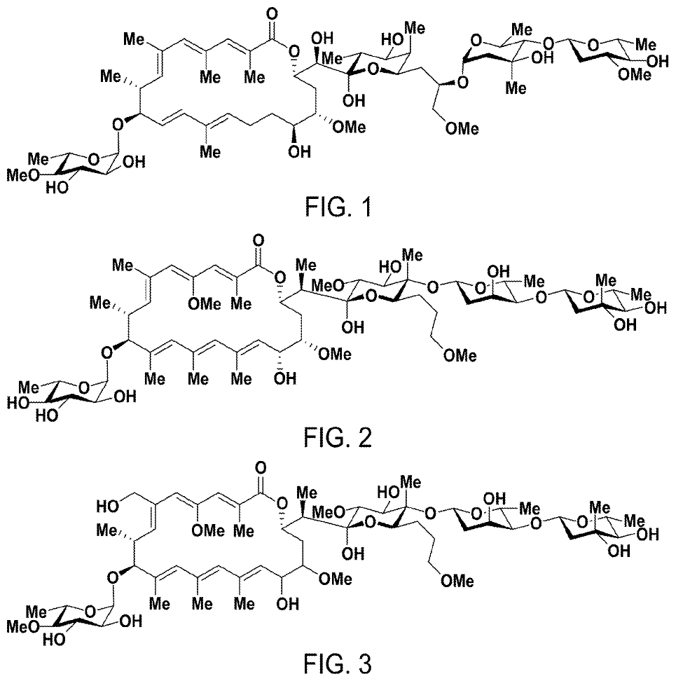

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Throughout the definitions, the term “Cₙ-Cₘ” indicates a range that includes the endpoints, wherein n and m are integers and indicate the number of carbons. Examples include, without limitation, C₁-C₄, C₁-C₆, and the like.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. In various aspects, the alkyl group contains from 1 to 24 carbon atoms (C₁-C₂₄), from 1 to 12 carbon atoms (C₁-C₁₂), from 1 to 10 carbon atoms (C₁-C₁₀), from 1 to 8 carbon atoms (C₁-C₈), from 1 to 6 carbon atoms (C₁-C₆), from 1 to 4 carbon atoms (C₁-C₄), from 1 to 3 carbon atoms (C₁-C₃), or 1 to 2 carbon atoms (C₁-C₂). The alkyl group can also be substituted or unsubstituted. Unless stated otherwise, the term “alkyl” contemplates both substituted and unsubstituted alkyl groups. The alkyl group can be substituted with one or more groups including, but not limited to, C₁-C₁₀alkoxy, C₁-C₁₀alkenyl, C₁-C₁₀alkynyl, C₃-C₁₀cycloalkyl, C₁-C₁₀heterocycloalkyl, C₆-C₁₈aryl, C₁-C₁₀heteroaryl, aldehyde, amino, carboxylic acid, oxo, halide, hydroxy, cyano, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, C₁-C₁₀alkyl, C₁-C₁₀alkoxy, C₁-C₁₀alkenyl, C₁-C₁₀alkynyl, C₃-C₁₀cycloalkyl, C₁-C₁₀heterocycloalkyl, C₆-C₁₈aryl, C₁-C₁₀heteroaryl, aldehyde, amino, carboxylic acid, halide, hydroxy, cyano, oxo, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, C₁-C₁₀alkyl, C₁-C₁₀alkoxy, C₁-C₁₀alkenyl, C₁-C₁₀alkynyl, C₃-C₁₀cycloalkyl, C₁-C₁₀heterocycloalkyl, C₆-C₁₈aryl, C₁-C₁₀heteroaryl, aldehyde, amino, carboxylic acid, halide, hydroxy, cyano, oxo, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Unless specified otherwise, the term “patient” refers to any mammalian animal, including but not limited to, humans.

As used herein, a “nucleoside analogue” is a compound that possess a capability to mimic native purine or pyrimidine nucleosides which can disrupt metabolic and regulatory pathways.

As used herein, an azanucleoside is a modified nucleoside in which one or more atoms in the furanosyl or aromatic ring has been replaced by a nitrogen atom. In some embodiments, the azanucleoside is a modified nucleoside having a modified cytosine, adenine, guanine, thymine, or uracil ring, wherein one or more carbon atoms in the ring has been replaced by a nitrogen atom. Modified nucleosides and azanucleoside, unless specified to the contrary, include prodrugs, for instances esters, phosphate esters, and phosphoramidates at the 5′ and/or 4′ and 3′ carbons.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Disclosed herein are compounds of Formula (1):

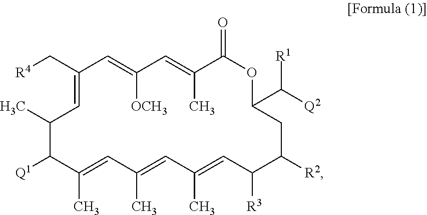

and pharmaceutically acceptable salts thereof,
wherein
R¹ is selected from H, OH, C₁₋₈alkyl, and OC₁₋₈alkyl, preferably CH₃.
R² is selected from H, OH, C₁₋₈alkyl, and OC₁₋₈alkyl, preferably OCH₃.
R³ is selected from H, OH, C₁₋₈alkyl, and OC₁₋₈alkyl, preferably H or OH.
R⁴ is selected from H, OH, C₁₋₈alkyl, and OC₁₋₈alkyl, preferably H or OH.
Q¹ is a group having the formula:

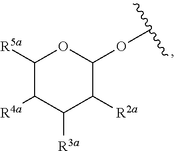

wherein
R^{2a} is selected from —R^{2a}*, —OR^{2a}*, OP(O)(OR^{2a}*)₂, OP(O)(OR^{2a}*)(N(R^{2a}*)₂, —N(R^{2a}*)₂, —N(R^{2a}*)₃, —C(O)R^{2a}*, —C(O)OR^{2a}*, —OC(O)R^{2a}*, —OC(O)OR^{2a}*, —NR^{2a}*C(O)R^{2a}*, —C(O)N(R^{2a}*)₂, NR^{2a}*C(O)OR^{2a}*, —OC(O)N(R^{2a}*)₂, —NR^{2a}*C(O)N(R^{2a}*)₂; —Cl, —F, —Br, —I, —NO₂, —CN, —N₃, —(OCH₂CH₂)ₘ—OR^{p};
R^{2a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{2a}* may be substituted one or more times by —OH, —COOH, —NH₂, —Cl, —F, —Br, —I, —NO₂, —CN, —N₃, PO(OH)₂, —(OCH₂CH₂)ₘ—OR^{p}; C₁₋₈heterocycyl, aryl, —OC₁₋₈heterocycyl, or C₁₋₈alkoxy,
wherein any two or more of R^{2a}* may together form a ring;
R^{3a} is selected from —R^{3a}*, —OR^{3a}*, OP(O)(OR^{3a}*)₂, OP(O)(OR^{3a}*)(N(R^{3a}*)₂, —N(R^{3a}*)₂, —N(R^{3a}*)₃—C(O)R^{3a}*, —C(O)OR^{3a}*, —OC(O)R^{3a}*, —OC(O)OR^{3a}*, —NR^{3a}*C(O)R^{3a}*, —C(O)N(R^{3a}*)₂, NR^{3a}*C(O)OR^{3a}*, —OC(O)N(R^{3a}*)₂, —NR^{3a}*C(O)N(R^{3a}*)₂; —Cl, —F, —Br, —I, —NO₂, —CN, —N₃, —(OCH₂CH₂)ₘ—OR^{p};
R^{3a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{3a}* may be substituted one or more times by —OH, —COOH, —NH₂, —Cl, —F, —Br, —I, —NO₂, —CN, —N₃, PO(OH)₂, —(OCH₂CH₂)ₘ—OR^{p}; C₁₋₈heterocycyl, aryl, —OC₁₋₈heterocycyl, or C₁₋₈alkoxy,
wherein any two or more of R^{3a}* may together form a ring;
R^{4a} is selected from —R^{4a}*, —OR^{4a}*, OP(O)(OR^{4a}*)₂, OP(O)(OR^{4a}*)(N(R^{4a}*)₂, —N(R^{4a}*)₂, —N(R^{4a}*)₃, —C(O)R^{4a}*, —C(O)OR^{4a}*, —OC(O)R^{4a}*, —OC(O)OR^{4a}*, —NR^{4a}*C(O)R^{4a}*, —C(O)N(R^{4a}*)₂, NR^{4a}*C(O)OR^{4a}*, —OC(O)N(R^{4a}*)₂, —NR^{4a}*C(O)N(R^{4a}*)₂; —Cl, —F, —Br, —I, —NO₂, —CN, —N₃, —(OCH₂CH₂)ₘ—OR^{p};
R^{4a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{4a}* may be substituted one or more times by —OH, —COOH, —NH₂, —Cl, —F, —Br, —I, —NO₂, —CN, —N₃, PO(OH)₂, —(OCH₂CH₂)ₘ—OR^{p}; C₁₋₈heterocycyl, aryl, —OC₁₋₈heterocycyl, or C₁₋₈alkoxy;
wherein any two or more of R^{4a}* may together form a ring;
R^{5a} is selected from —R^{5a}*, —OR^{5a}*, OP(O)(OR^{5a}*)₂, OP(O)(OR^{5a}*)(N(R^{5a}*)₂, —N(R^{5a}*)₂, —N(R^{5a}*)₃, —C(O)R^{5a}*, —C(O)OR^{5a}*, —OC(O)R^{5a}*, —OC(O)OR^{5a}*, —NR^{5a}*C(O)R^{5a}*, —C(O)N(R^{5a}*)₂, NR^{5a}*C(O)OR^{5a}*, —OC(O)N(R^{5a}*)₂, —NR^{5a}*C(O)N(R^{5a}*)₂; —Cl, —F, —Br, —I, —NO₂, —CN, —N₃, —(OCH₂CH₂)ₘ—OR^{p};
R^{5a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{5a}* may be substituted one or more times by —OH, —COOH, —NH₂, —Cl, —F, —Br, —I, —NO₂, —CN, —N₃, PO(OH)₂, —(OCH₂CH₂)ₘ—OR^{p}; C₁₋₈heterocycyl, aryl, —OC₁₋₈heterocycyl, or C₁₋₈alkoxy;
wherein R^{p} is in each case selected from H, C₁₋₁₀alkyl, and aryl;
wherein any two or more of R^{5a}* may together form a ring;
wherein any two of more of R^{2a}, R^{3a}, R^{4a}, and R^{5a} may together form a ring;
Q² is a group having the formula:

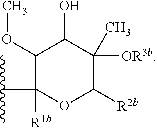

wherein:
R^{1b} is selected from H, OH, C₁₋₈alkyl, and OC₁₋₈alkyl;
R^{2b} is selected from —R^{2b}*, —OR^{2b}*, OP(O)(OR^{2b}*)₂, OP(O)(OR^{2b}*)(N(R^{2b}*)₂, —N(R^{2b}*)₂, —N(R^{2b}*)₃, —C(O)R^{2b}*, —C(O)OR^{2b}*, —OC(O)R^{2b}*, —OC(O)OR^{2b}*, —NR^{2b}*C(O)R^{2b}*, —C(O)N(R^{2b}*)₂, NR^{2b}*C(O)OR^{2b}*, —OC(O)N(R^{2b}*)₂, —NR^{2b}*C(O)N(R^{2b}*)₂; —Cl, —F, —Br, —I, —NO₂, —CN, —N₃, —(OCH₂CH₂)ₘ—OR^{p};
R^{2b}* is in each case independently selected from H, C₁₋₈alkyl, C₁₋₈alkenyl, C₁₋₁₀alkynyl, aryl, C₃₋₈cyclolkyl, C₃₋₈cycloalkenyl, C₇₋₁₀cycloalkynyl, aryl, C₁₋₈heterocycyl, C₃₋₈heteroaryl; wherein each R^{2b}* may be substituted one or more times by —OH, —COOH, —NH₂, —Cl, —F, —Br, —I, —NO₂, —CN, —N₃, PO(OH)₂, —(OCH₂CH₂)ₘ—OR^{p}; C₁₋₈heterocycyl, aryl, —OC₁₋₈heterocycyl, or C₁₋₈alkoxy,
wherein any two or more of R^{2b}* may together form a ring;
R^{3b} is selected from H or a group having the formula:

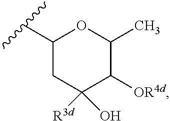

wherein
R^{3d} is selected from H and CH₃; and
R^{4d} is selected from H and a group having the formula:

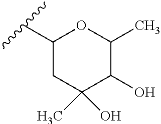 [SMILES: *C1CC(C)(O)C(O)C(C)O1]

In some embodiments, the compound can be a compound of Formula (2a), Formula (2b), or Formula (2c):

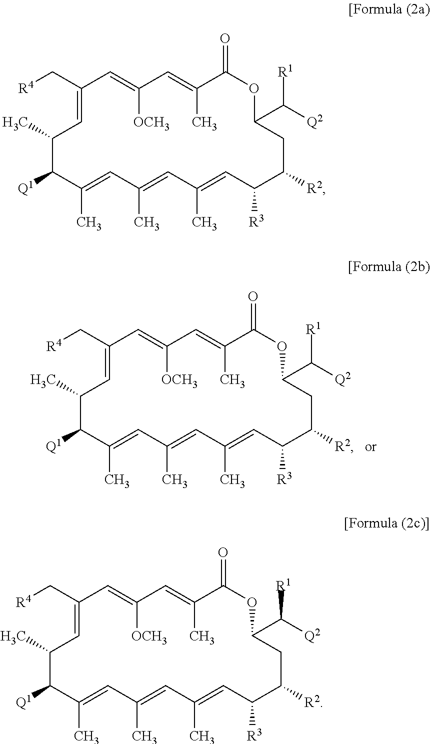

In some embodiments, Q¹ is an (L) sugar having the conformation.

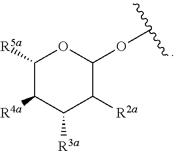

Exemplary stereoisomers for Q¹ include

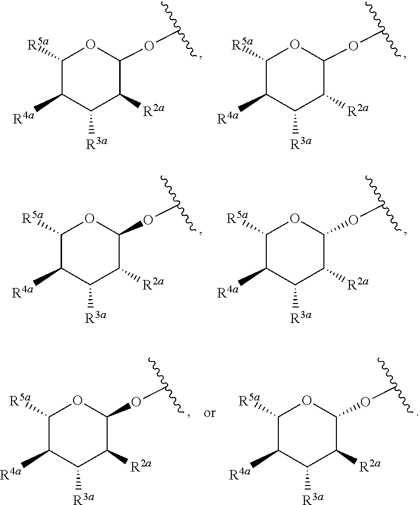

Q² can have the formula:

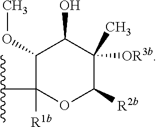

The compound according to any preceding claim, wherein Q² can have either formula:

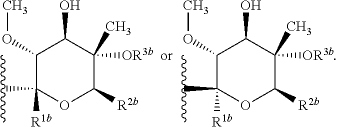

R^{3b} can be group having the formula:

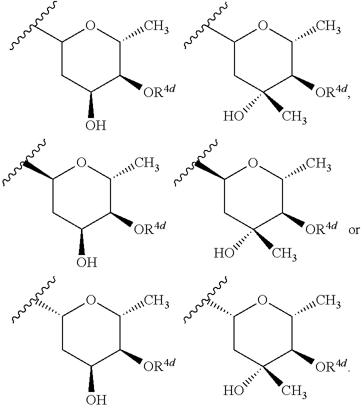

R^{4d} can be a group having the formula:

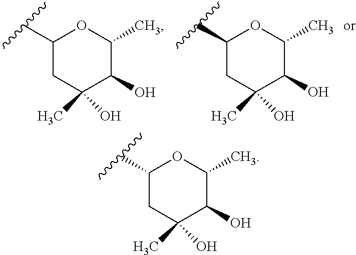

The compounds disclosed herein are preferably characterized when R^{5a} is selected from CH₃ and CH₂OH, most preferably CH₃.

In some embodiments, the compounds are derivatized at the R^{3a} position, and R^{4a} and R^{2a} are each OH. In other embodiments, the compounds are derivatized at the R^{4a} position, and R^{3a} and R^{2a} are each OH. In further embodiments the compounds are derivatized at the R^{2a} position, and R^{3a} and R^{4a} are each OH. Other embodiments in which both of R^{4a} and R^{2a} are derivatized, both of R^{3a} and R^{2a} are derivatized, or both of R^{4a} and R^{3a} are derivatized are also contemplated. Further embodiments when all of R^{2a}, R^{3a}, and R^{4a} are derivatized as also within the scope of the disclosure. It is contemplated that for all of these embodiments, R^{2b} may be as naturally found in ammocidin (—CH₂CH₂CH₂OCH₃), but the disclosed compounds may also be derivatized at this position as well. In yet other embodiments, none of R^{2a}, R^{3a}, and R^{4a} are derivatized (i.e., all are OH), but R^{2b} is not —CH₂CH₂CH₂OCH₃.

The macrolide derivatives disclosed herein may be characterized wherein at least one of R^{2a}, R^{3a}, and R^{4a} is not OH, or R^{2b} is not —CH₂CH₂CH₂OCH₃.

In certain embodiments, the macrolide derivatives can include an azide, tetrazine, cyclooctyne, or trans-cyclooctene group. Such groups are useful in “click” cycloaddition reactions which may be used to further elaborate the macrolide analog. Suitable cyclooctynes include bicyclo[6.1.0]nonyne (“BCN”), dibenzocyclooctyne, dibenzocyclooctyne-amine, and substituted derivatives of each of these groups. Exemplary groups include the following:

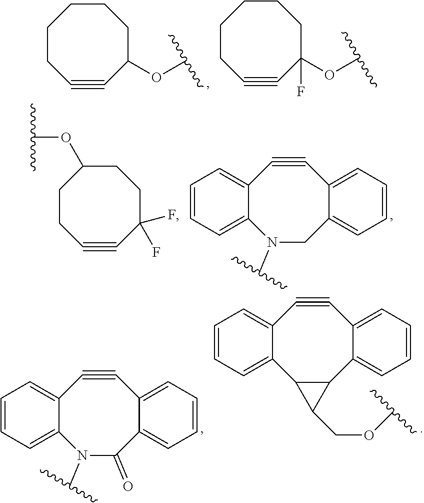

In certain embodiments, one or more hydroxyl group on the naturally occurring (L)-fucose moiety may be modified. As used herein, modifications include oxidation, stereochemical inversion, and/or functionalization via chemical reaction. These compounds may be accessed by exploiting the different chemical reactivity of each hydroxyl in the fucose ring. Generally, the R^{3a} position is most reactive to electrophiles, and this position may selectively be modified. In order to modify at other positions, it may be necessary to first protect the R^{3a} hydroxyl (as well as other positions). Such techniques are known to those in the art.

In some embodiments, the macrolide derivatives can include a mitochondrial targeting moiety. Exemplary moieties include quaternary phosphonium and ammonium ions, for instance triphenylphosphonium, trialkylammonium (e.g., trimethylammonium, triethylammonium) guanidinium, (including both cyclic and acyclic guanidiniums), pyridinium, rhodamines, dequaliniums, (E)-4-(1H-Indol-3-ylvinyl)-N-methylpyridinium iodide, and the like.

In some embodiments, R^{3a} can be —OR^{3a}*, —OC(O)R^{3a}*, OP(O)(OR^{3a}*)₂, OP(O)(OR^{3a}*)(N(R^{3a}*)₂, —OC(O)OR^{3a}, —OC(O)N(R^{3a}*)₂, or —(OCH₂CH₂)ₘ—OR^{3a}* (m being from 1-100). In each case, R^{3a}* is independently selected from H (except when R^{3a} is OR^{3a}*), C₁₋₈alkyl, C₁₋₈alkenyl, or C₁₋₁₀alkynyl, C₇₋₁₀cyclolkynyl, C₇₋₁₀cycloalkenyl, C₁₋₈heterocyclyl, C₁₋₈alkylC₇₋₁₀cyclolkynyl, C₁₋₈alkylC₇₋₁₀cycloalkenyl, or C₁₋₈alkylC₁₋₈heterocyclyl. Such R^{3a}* groups may be further substituted one or more times; exemplary substituents include COOR, F, Cl, Br, I, SO₃R, OSO₃R, —PPh₃, —CH₂NEt₃, —CH₂NMe₃, P(O)(OR)₂, OP(O)(OR)₂, N₃, heterocyclyl, heteroaryl, aryl, cycloalkyl, wherein R is in each case independently selected from H, alkyl, and cycloalkyl.

In certain embodiments R^{3a} is derivatized as an ether or ester, e.g., —OR^{3a}* or —OC(O)R^{3a}*, and others derivatized as phosphonate or phosphoramidate product, e.g., —OP(O)(OR^{3a}*)₂ or OP(O)(OR^{3a}*)(N(R^{3a}*)₂, wherein R^{3a} is preferably selected from H, aryl, and C₁₋₈alkyl, or is derived from an amino acid, e.g.:

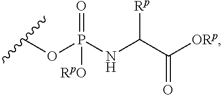

wherein R^{p} is as defined above.

In some embodiments, R^{3a}* can be a moiety having the formula:

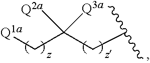

wherein the sum of z and z′ is no greater than 7, Q^{2a} and Q^{3a} are each hydrogen or together form a C₁₋₈heterocycyl, and Q^{1a} is selected from COOH, aryl, —C≡CH, —CF₃, N₃, C₇₋₁₀cyclolkynyl, C₇₋₁₀ cycloalkenyl, C₁₋₁₀heteroaryl, or C₁₋₁₀heterocycyl. Particularly preferred Q^{1a} groups include COOH, N₃ and CF₃. In other embodiments, Q^{1a} is selected from aryl and C₁₋₁₀heteroaryl, optionally substituted one or more times as described above. In other embodiments, Q^{1a} is a dibenzocyclooctyne, dibenzocyclooctyne-amine, or trans-cyclooctene.

In certain embodiments, Q^{1a} can be a moiety having the formula:

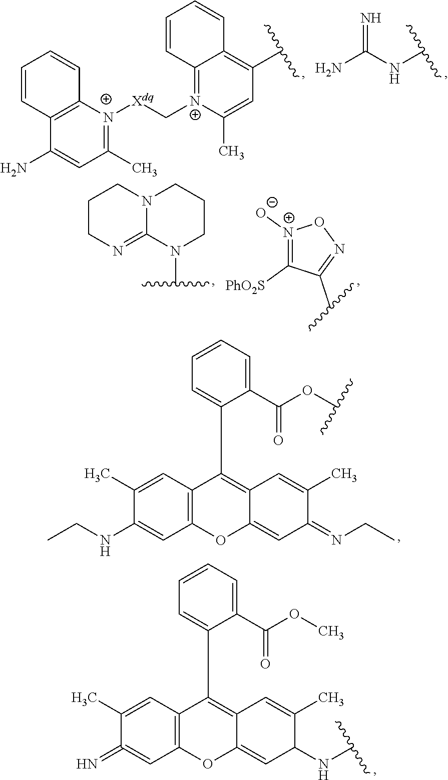

wherein X^{dq} is an alkylene chain having from 2-12 CH₂ units, preferably 5-12 CH₂ units, more preferably 7-11 CH₂ units, and especially preferably 8-10 CH₂ units. The skilled person will recognize that while some of the above moieties are depicted in electronically neutral form, these moieties can be protonated and paired with an appropriate counterion. Likewise, the quinolinium cation depicted above will be accompanied by a charge balancing ion or ions, for example dichloride, dibromide, diiodide, diacetate, and the like.

In some embodiments, R^{2a} can be —OR^{2a}*, —OC(O)R^{2a}*, OP(O)(OR^{2a}*)₂, OP(O)(OR^{2a}*)(N(R^{2a}*)₂, —OC(O)OR^{2a}, —OC(O)N(R^{2a}*)₂, or —(OCH₂CH₂)ₘ—OR^{2a}* (m being from 1-100). In each case, R^{2a}* is independently selected from H (except when R^{2a} is OR^{2a}*), C₁₋₈alkyl, C₁₋₈alkenyl, or C₁₋₁₀alkynyl, C₇₋₁₀cyclolkynyl, C₇₋₁₀cycloalkenyl, C₁₋₈heterocyclyl, C₁₋₈alkylC₇₋₁₀cyclolkynyl, C₁₋₈alkylC₇₋₁₀cycloalkenyl, or C₁₋₈alkylC₁₋₈heterocyclyl. Such R^{2a}* groups may be further substituted one or more times; exemplary substituents include COOR, F, Cl, Br, I, SO₃R, OSO₃R, —PPh₃, —CH₂NEt₃, —CH₂NMe₃, P(O)(OR)₂, OP(O)(OR)₂, N₃, heterocyclyl, heteroaryl, aryl, cycloalkyl, wherein R is in each case independently selected from H, alkyl, and cycloalkyl.

In certain embodiments R^{2a} is derivatized as an ether or ester, e.g., —OR^{2a}* or —OC(O)R^{2a}*, and others derivatized as phosphonate or phosphoramidate product, e.g., —OP(O)(OR^{2a}*)₂ or OP(O)(OR^{2a}*)(N(R^{2a}*)₂, wherein R^{2a} is preferably selected from H, aryl, and C₁₋₈alkyl, or is derived from an amino acid, e.g.:

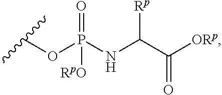

wherein R^{p} is as defined above.

In some embodiments, R^{2a}* can be a moiety having the formula:

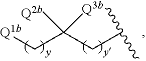

wherein the sum of y and y′ is no greater than 7, Q^{2b} and Q^{3b} are each hydrogen or together form a C₁₋₈heterocycyl, and Q^{1b} is selected from COOH, aryl, —C≡CH, —CF₃, N₃, C₇₋₁₀cyclolkynyl, C₇₋₁₀cycloalkenyl, C₁₋₁₀heteroaryl, or C₁₋₁₀heterocycyl. Particularly preferred Q^{1b} groups include COOH, N₃ and CF₃. In other embodiments, Q^{1b} is selected from aryl and C₁₋₁₀heteroaryl, optionally substituted one or more times as described above. In other embodiments, Q^{1b} is a dibenzocyclooctyne, dibenzocyclooctyne-amine, or trans-cyclooctene.

In certain embodiments, Q^{1b} can be a moiety having the formula:

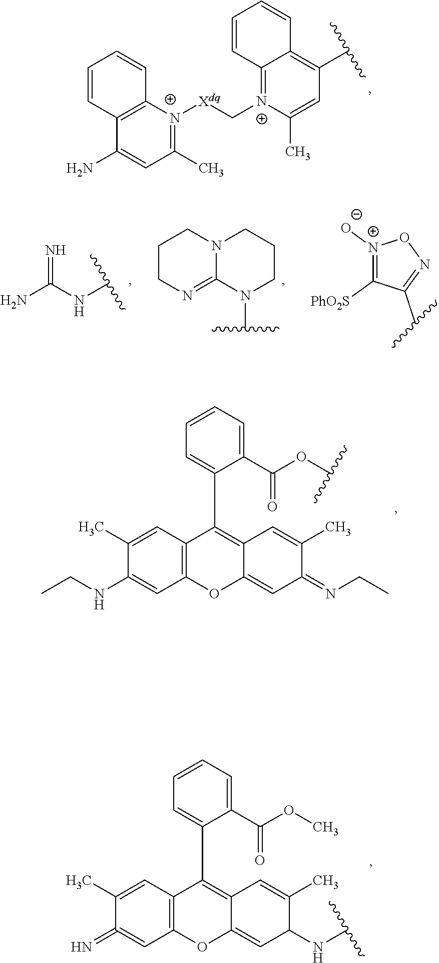

wherein X^{dq} is an alkylene chain having from 2-12 CH₂ units, preferably 5-12 CH₂ units, more preferably 7-11 CH₂ units, and especially preferably 8-10 CH₂ units. The skilled person will recognize that while some of the above moieties are depicted in electronically neutral form, these moieties can be protonated and paired with an appropriate counterion. Likewise, the quinolinium cation depicted above will be accompanied by a charge balancing ion or ions, for example dichloride, dibromide, diiodide, diacetate, and the like.

In some embodiments, R^{4a} can be —OR^{4a}*, —OC(O)R^{4a}*, OP(O)(OR^{4a}*)₂, OP(O)(OR^{4a}*)(N(R^{4a}*)₂, —OC(O)OR^{4a}, —OC(O)N(R^{4a}*)₂, or —(OCH₂CH₂)ₘ—OR^{4a}* (m being from 1-100). In each case, R^{4a}* is independently selected from H (except when R^{4a} is OR^{4a}*), C₁₋₈alkyl, C₁₋₈alkenyl, or C₁₋₁₀alkynyl, C₇₋₁₀cyclolkynyl, C₇₋₁₀cycloalkenyl, C₁₋₈heterocyclyl, C₁₋₈alkylC₇₋₁₀cyclolkynyl, C₁₋₈alkylC₇₋₁₀cycloalkenyl, or C₁₋₈alkylC₁₋₈heterocyclyl. Such R^{4a}* groups may be further substituted one or more times; exemplary substituents include COOR, F, Cl, Br, I, SO₃R, OSO₃R, —PPh₃, —CH₂NEt₃, —CH₂NMe₃, P(O)(OR)₂, OP(O)(OR)₂, N₃, heterocyclyl, heteroaryl, aryl, cycloalkyl, wherein R is in each case independently selected from H, alkyl, and cycloalkyl.

In certain embodiments R^{4a} is derivatized as an ether or ester, e.g., —OR^{4a}* or —OC(O)R^{4a}*, and others derivatized as phosphonate or phosphoramidate product, e.g., —OP(O)(OR^{4a}*)₂ or OP(O)(OR^{4a}*)(N(R^{4a}*)₂, wherein R^{4a} is preferably selected from H, aryl, and C₁₋₈alkyl, or is derived from an amino acid, e.g.:

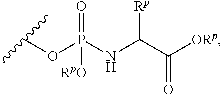

wherein R^{p} is as defined above.

In some embodiments, R^{4a}* can be a moiety having the formula:

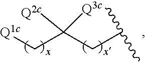

wherein the sum of x and x′ is no greater than 7, Q^{2c} and Q^{3c} are each hydrogen or together form a C₁₋₈heterocycyl, and Q^{1c} is selected from COOH, aryl, —C≡CH, —CF₃, N₃, C₇₋₁₀cyclolkynyl, C₇₋₁₀cycloalkenyl, C₁₋₁₀heteroaryl, or C₁₋₁₀heterocycyl. Particularly preferred Q^{1c} groups include COOH, N₃ and CF₃. In other embodiments, Q^{1c} is selected from aryl and C₁₋₁₀heteroaryl, optionally substituted one or more times as described above. In other embodiments, Q^{1c} is a dibenzocyclooctyne, dibenzocyclooctyne-amine, or trans-cyclooctene.

In certain embodiments, Q^{1c} can be a moiety having the formula:

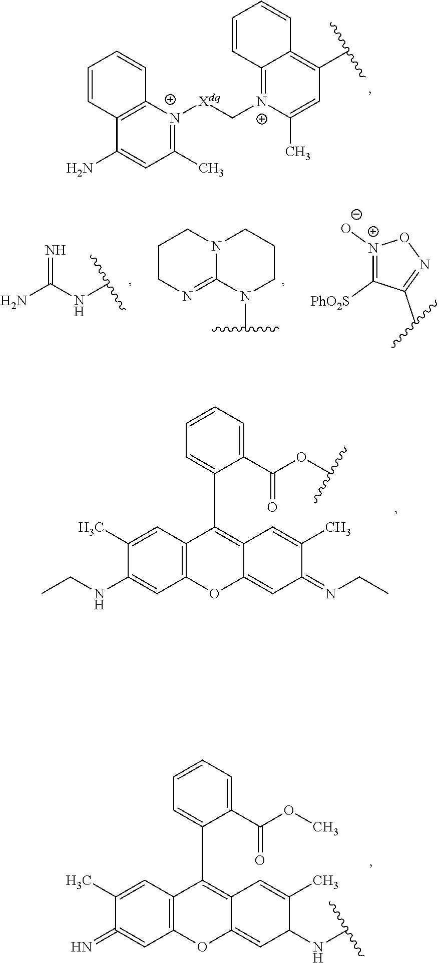

Wherein X^{dq} is an alkylene chain having from 2-12 CH₂ units, preferably 5-12 CH₂ units, more preferably 7-11 CH₂ units, and especially preferably 8-10 CH₂ units. The skilled person will recognize that while some of the above moieties are depicted in electronically neutral form, these moieties can be protonated and paired with an appropriate counterion. Likewise, the quinolinium cation depicted above will be accompanied by a charge balancing ion or ions, for example dichloride, dibromide, diiodide, diacetate, and the like.

In some embodiments, the macrolide derivatives can be modified at the R^{2b} position, for instance by modification of the fermentation conditions as described herein. R^{2b} can be —OR^{2b}*, —OC(O)R^{2b}*, —PPh₃, —CH₂NEt₃, —CH₂NMe₃, OP(O)(OR^{2b}*)₂, OP(O)(OR^{2b}*)(N(R^{2b}*)₂, —OC(O)OR^{2b}, —OC(O)N(R^{2b}*)₂, or —(OCH₂CH₂)ₘ—OR^{2b}* (m being from 1-100). In each case, R^{2b}* is independently selected from H (except when R^{2b} is OR^{2b}*), C₁₋₈alkyl, C₁₋₈alkenyl, or C₁₋₁₀alkynyl, C₇₋₁₀cyclolkynyl, C₇₋₁₀cycloalkenyl, C₁₋₈heterocyclyl, C₁₋₈alkylC₇₋₁₀cyclolkynyl, C₁₋₈alkylC₇₋₁₀cycloalkenyl, or C₁₋₈alkylC₁₋₈heterocyclyl. Such R^{2b}* groups may be further substituted one or more times; exemplary substituents include COOR, F, Cl, Br, I, SO₃R, OSO₃R, P(O)(OR)₂, OP(O)(OR)₂, N₃, C₁₋₁₀heterocyclyl, C₁₋₁₀heteroaryl, C₆₋₁₈aryl, C₃₋₁₀cycloalkyl, wherein R is in each case independently selected from H, alkyl, and cycloalkyl.

In certain embodiments R^{2b} is derivatized as an ether or ester, e.g., —OR^{2b}* or —OC(O)R^{2b}*, and others derivatized as phosphonate or phosphoramidate product, e.g., —OP(O)(OR^{2b}*)₂ or OP(O)(OR^{2b}*)(N(R^{2b}*)₂, wherein R^{2b} is preferably selected from H, aryl, and C₁₋₈alkyl, or is derived from an amino acid, e.g.:

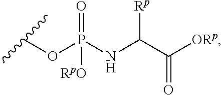

wherein R^{p} is as defined above.

In some embodiments, R^{2b}* can be a moiety having the formula:

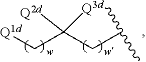

wherein the sum of w and w′ is no greater than 7, Q^{2d} and Q^{3d} are each hydrogen or together form a C₁₋₈heterocycyl, and Q^{1d} is selected from COOH, aryl, —C≡CH, —CF₃, N₃, C₇₋₁₀cyclolkynyl, C₇₋₁₀cycloalkenyl, C₁₋₁₀heteroaryl, or C₁₋₁₀heterocycyl. Particularly preferred Q^{1d} groups include COOH, N₃ and CF₃. In other embodiments, Q^{1d} is selected from aryl and C₁₋₁₀heteroaryl, optionally substituted one or more times as described above. In other embodiments, Q^{1d} is a dibenzocyclooctyne, dibenzocyclooctyne-amine, or trans-cyclooctene.

In certain embodiments, Q^{1d} can be a moiety having the formula:

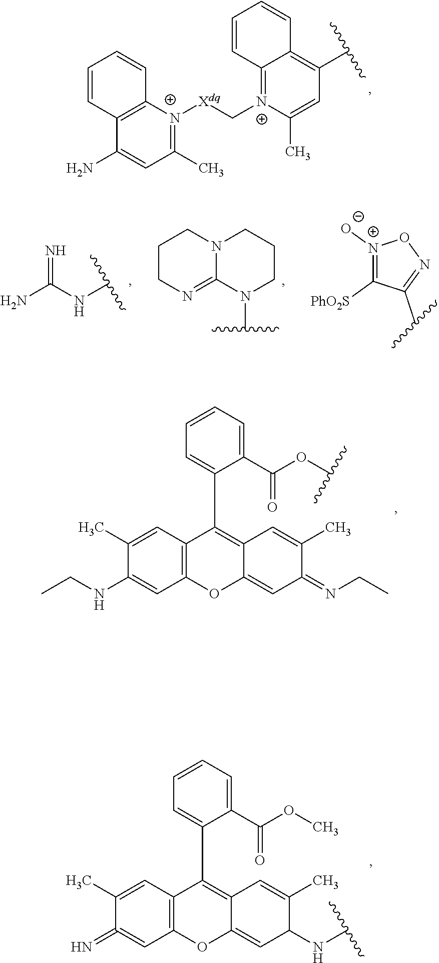

wherein X^{dq} is an alkylene chain having from 2-12 CH₂ units, preferably 5-12 CH₂ units, more preferably 7-11 CH₂ units, and especially preferably 8-10 CH₂ units. The skilled person will recognize that while some of the above moieties are depicted in electronically neutral form, these moieties can be protonated and paired with an appropriate counterion. Likewise, the quinolinium cation depicted above will be accompanied by a charge balancing ion or ions, for example dichloride, dibromide, diiodide, diacetate, and the like.

Also disclosed are compounds of Formula (3a), (3b), and (3c):

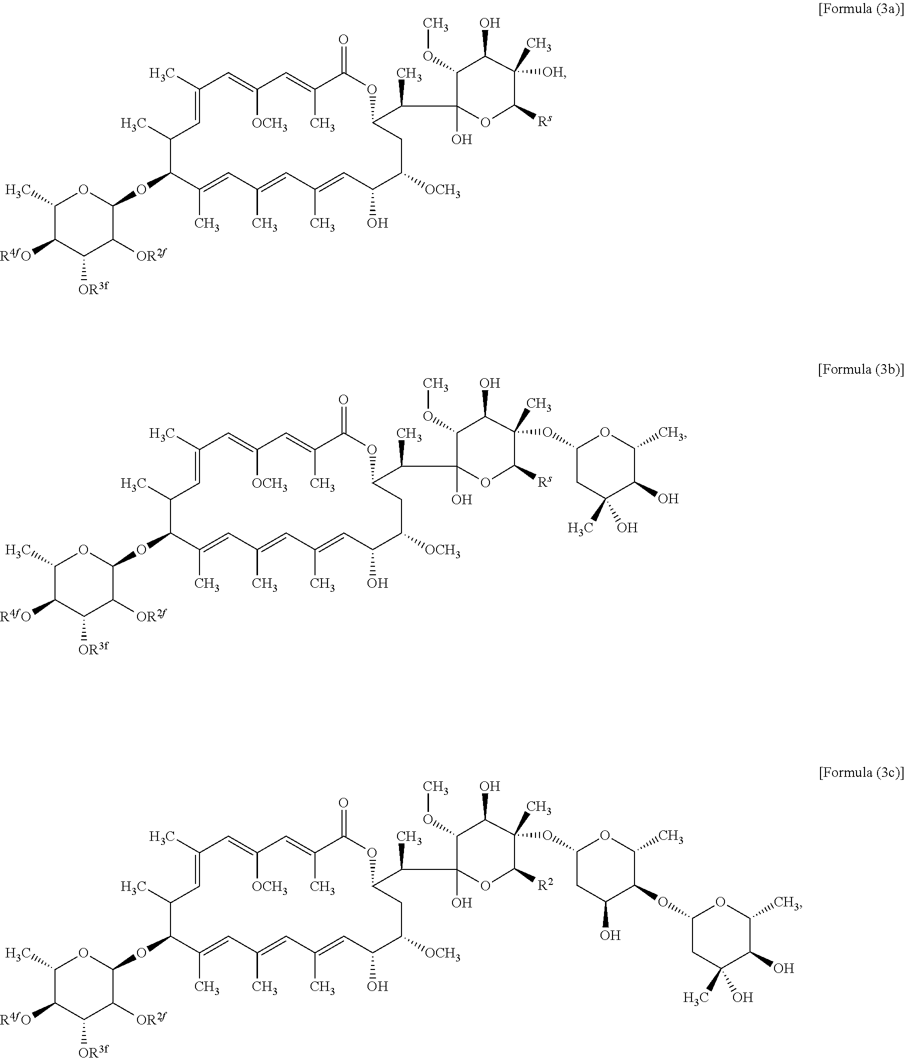

wherein R^{2f} is —R^{f}*, —C(O)R^{f}*, —C(O)OR^{f}*, —OC(O)R^{2b}*, —OC(O)OR^{f}*, C(O)N(R^{f}*)₂, or —(OCH₂CH₂)ₘ—OR^{f}*;
wherein R^{3f} is —R^{f}*, —C(O)R^{f}*, —C(O)OR^{f}*, —OC(O)R^{2b}*, —OC(O)OR^{f}*, C(O)N(R^{f}*)₂, or —(OCH₂CH₂)ₘ—OR^{f}*;
wherein R^{4f} is —R^{f}*, —C(O)R^{f}*, —C(O)OR^{f}*, —OC(O)R^{2b}*, —OC(O)OR^{f}*, C(O)N(R^{f}*)₂, or —(OCH₂CH₂)ₘ—OR^{f}*;
R^{f}* is in each case independently selected from H, substituted or unsubstituted C₁₋₈alkyl, substituted or unsubstituted C₁₋₈alkenyl, substituted or unsubstituted C₁₋₁₀alkynyl, substituted or unsubstituted aryl, substituted or unsubstituted C₃₋₈cyclolkyl, substituted or unsubstituted C₃₋₈cycloalkenyl, substituted or unsubstituted C₇₋₁₀cycloalkynyl, aryl, substituted or unsubstituted C₁₋₈heterocycyl, substituted or unsubstituted C₃₋₈heteroaryl;
R^{s} is selected from H, substituted or unsubstituted C₁₋₈alkyl, substituted or unsubstituted C₁₋₈alkenyl, substituted or unsubstituted C₁₋₁₀alkynyl, substituted or unsubstituted aryl, substituted or unsubstituted C₃₋₈cyclolkyl, substituted or unsubstituted C₃₋₈cycloalkenyl, substituted or unsubstituted C₇₋₁₀cycloalkynyl, substituted or unsubstituted aryl, C₁₋₈heterocycyl, substituted or unsubstituted C₃₋₈heteroaryl;
provided that R^{2f}, R^{3f} and R^{4f} are not simultaneously H when R^{s} is CH₂CH₂CH₂OCH₃.

As used herein, a substituted group is one in which one or more hydrogen atoms that is bound to a carbon atom is replaced with a non-hydrogen group. Exemplary non-hydrogen groups include halo (F, Cl, Br, I), hydroxyl, sulfhydryl, C₁-C₂₄ alkoxy, C₅-C₂₄ aryloxy, acyl (including C₂-C₂₄ alkylcarbonyl (—CO-alkyl) and C₆-C₂₄ arylcarbonyl (—CO—C₅-C₂₄ aryl)), haloacyl (including C₂-C₂₄ haloalkylcarbonyl (—C)-haloalkyl) and C₆-C₂₄ haloarylcarbonyl (—CO-aryl)), C₂-C₂₄ thioacyloxy (including —O—(CS)-alkyl and O—(CS)-aryl), C₂-C₂₄ thiohaloacyloxy (including O—(CS)-haloalkyl and O—(CS)-haloaryl)), acyloxy (—O-acyl), C₂-C₂₄ alkoxycarbonyl (—(CO)—O-alkyl), C₆-C₂₄ aryloxycarbonyl (—(CO)—O-aryl), C₇-C₂₄ alkaryloxycarbonyl (—(CO)—O-alkaryl), C₇-C₂₄ aralkyloxycarbonyl (—(CO)—O— aralkyl), C₂-C₂₄ haloalkylcarbonato (—O—(CO)—O-haloalkyl)), C₆-C₂₄ haloarylcarbonato (O—(CO)—O-haloaryl), C₂-C₂₄ alkylthiocarbonato (—O—(CS)—O-alkyl)), C₆-C₂₄ arylthiocarbonato (—O—(CS)—O-aryl), C₂-C₂₄ haloalkylthiocarbonato (—O—(CS)—O-haloalkyl), and C₆-C₂₄ haloarylthiocarbonato (—O—(CS)—O-haloaryl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N—(C₁-C₂₄ alkyl), N—(C₆-C₂₄ aryl)-substituted carbamoyl, thiocarbamoyl (—(CS)—NH₂), carbamido (—NH—(CO)—NH₂), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonamido (—SO₂NH₂), sulfonato (—SO₂O—), C₁-C₂₄ alkylsulfanyl (—S-alkyl; also termed “alkylthio”), arylsulfanyl (—S-aryl; also termed “arylthio”), C₁-C₂₄ alkylsulfinyl ((SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₄ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl).

In some embodiments, R^{2f} is —C(O)R^{2f}*, and R^{3f} and R^{4f} are each H and R^{s} is CH₂CH₂CH₂OCH₃. Exemplary groups for R^{2f}* include substituted or unsubstituted C₁₋₈alkyl or substituted or unsubstituted C₃₋₈heteroaryl. Exemplary C₃₋₈heteroaryl include pyridin-2-yl, pyridin-3-yl, and pyridin-4-yl. Preferred C₁₋₈alkyl include methyl, ethyl, isobutyl, and isopentyl. Preferred substituent groups include amino, carboxy, and sulfhydryl.

In some embodiments, R^{2f} has the structure:

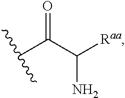

wherein R^{aa} is H, CH₃, CH(CH₃)₂, CH₂-phenyl, CH₂-4-(hydroxyphenyl), CH₂-(1H-imidazol-4-yl), CH₂-(1H-indol-3-yl), CH₂CH(CH₃)₂, CH₂OH, CH(OH)CH₃, CH₂SH, CH₂COOH, CH₂CH₂COOH, CH₂CONH₂, CH₂CH₂CONH₂, (CH₂)₄NH₂, CH₂CH₂CH₂NHC(═NH)NH₂, or CH₂CH₂SCH₃.

In other embodiments, R^{3f} is —C(O)R^{f}*, and R^{2f} and R^{4f} are each H and R^{s} is CH₂CH₂CH₂OCH₃. Exemplary groups for R^{2f}* include substituted or unsubstituted C₁₋₈alkyl or substituted or unsubstituted C₃₋₈heteroaryl. Exemplary C₃₋₈heteroaryl include pyridin-2-yl, pyridin-3-yl, and pyridin-4-yl. Preferred C₁₋₈alkyl include methyl, ethyl, isobutyl, and isopentyl. Preferred substituent groups include amino, carboxy, and sulfhydryl.

In some embodiments, R^{3f} has the structure:

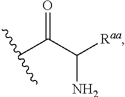

wherein R^{aa} is H, CH₃, CH(CH₃)₂, CH₂-phenyl, CH₂-4-(hydroxyphenyl), CH₂-(1H-imidazol-4-yl), CH₂-(1H-indol-3-yl), CH₂CH(CH₃)₂, CH₂OH, CH(OH)CH₃, CH₂SH, CH₂COOH, CH₂CH₂COOH, CH₂CONH₂, CH₂CH₂CONH₂, (CH₂)₄NH₂, CH₂CH₂CH₂NHC(═NH)NH₂, or CH₂CH₂SCH₃.

In further embodiments, R^{4f} is —C(O)R^{f}*, and R^{2f} and R^{3f} are each H and R^{s} is CH₂CH₂CH₂OCH₃. Exemplary groups for R^{2f}* include substituted or unsubstituted C₁₋₈alkyl or substituted or unsubstituted C₃₋₈heteroaryl. Exemplary C₃₋₈heteroaryl include pyridin-2-yl, pyridin-3-yl, and pyridin-4-yl. Preferred C₁₋₈alkyl include methyl, ethyl, isobutyl, and isopentyl. Preferred substituent groups include amino, carboxy, and sulfhydryl.

In some embodiments, R^{4f} has the structure:

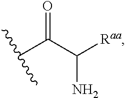

wherein R^{aa} is H, CH₃, CH(CH₃)₂, CH₂-phenyl, CH₂-4-(hydroxyphenyl), CH₂-(1H-imidazol-4-yl), CH₂-(1H-indol-3-yl), CH₂CH(CH₃)₂, CH₂OH, CH(OH)CH₃, CH₂SH, CH₂COOH, CH₂CH₂COOH, CH₂CONH₂, CH₂CH₂CONH₂, (CH₂)₄NH₂, CH₂CH₂CH₂NHC(═NH)NH₂, or CH₂CH₂SCH₃.

In certain embodiments, R^{f}* is selected from:

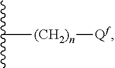

wherein n is 0, 1, 2, 3, 4, 6, 7, 8, 9, or 10, preferably 0, 1, 2, 3, or 4 and Q^{f} is CF₃, —C≡CH, N₃, —C≡N, COOH, (CO)—O-alkyl, aryl (e.g., 4-fluorophenyl, 3-fluorophenyl, 2-fluorophenyl), adamantyl, C₁₋₁₀heterocyclyl, substituted or unsubstituted C₇₋₁₀cycloalkynyl. In some embodiments, n is 0, while in other embodiments, n is 3. In certain embodiments, n is 2, 3, or 4, and Q^{f} is a C₁₋₁₀heterocyclyl having the formula:

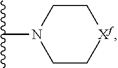

wherein X^{f} is null, CH₂, O, S, or NR^{xf}, wherein R^{f} is H or C₁₋₃alkyl, preferably methyl.

In certain embodiments, R^{f}* is (CO)—O-alkyl, wherein the alkyl is methyl or ethyl.

In some embodiments, R^{s} is selected from

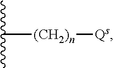

wherein n is 0, 1, 2, 3, 4, 6, 7, 8, 9, or 10, and Q^{s} is CF₃, —PPh₃, —CH₂NEt₃, —CH₂NMe₃, —C≡CH, N₃, —C≡N, COOH, (CO)—O-alkyl, aryl (e.g., 4-fluorophenyl, 3-fluorophenyl, 2-fluorophenyl), adamantyl, C₁₋₈heterocyclyl, substituted or unsubstituted C₇₋₁₀cycloalkynyl.

In certain embodiments, Q^{f} can be a moiety having the formula:

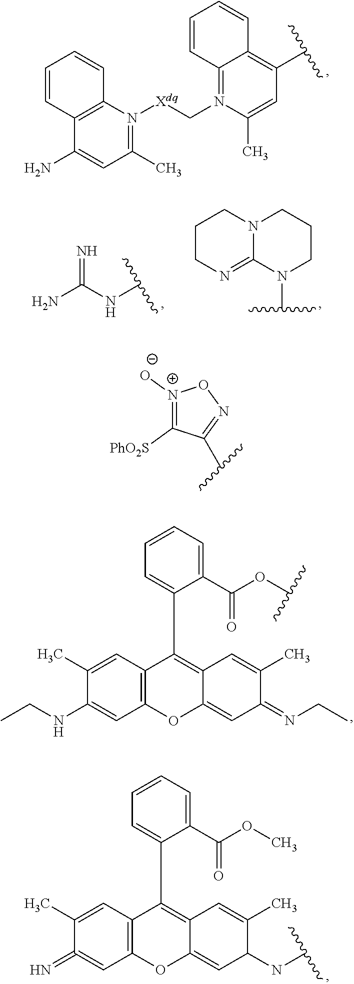

wherein X^{dq} is an alkylene chain having from 2-12 CH₂ units, preferably 5-12 CH₂ units, more preferably 7-11 CH₂ units, and especially preferably 8-10 CH₂ units. The skilled person will recognize that while some of the above moieties are depicted in electronically neutral form, these moieties can be protonated and paired with an appropriate counterion. Likewise, the quinolinium cation depicted above will be accompanied by a charge balancing ion or ions, for example dichloride, dibromide, diiodide, diacetate, and the like. The biosynthesis of naturally occurring apoptolidin and related macrolides by Nocardiopsis sp. FU40 is initiated with (R)-methoxymalonate, which is a substrate for the (R)-2-methoxymalonyl-acyl carrier protein (“MeOM-ACP”). This protein is encoded in the five gene contiguous gene cassette (apoK-M2), which can be disrupted by double gene replacement of apoJK, completely abolishing production of all apoptolidin macrolides. Fermentation with mutant Nocardiopsis sp. FU40 apoJK::aac(3)IV with N-acetylcysteamine, (NAC) thioester of (R)-2-methoxymalonate restores apoptolidin biosynthesis. Fermentation of Nocardiopsis sp. FU40 apoJK::aac(3)IV with a thioester of formula R^{2b}—C(O)SR^{st} or R^{s}—C(O)SR^{st} (wherein R^{st} is an alkyl or aryl group) produces macrolides with the corresponding R^{2b} or R^{s} groups.

As used herein, the phrase “therapeutic agent” refers to any agent that, when administered to a subject, has a therapeutic and/or diagnostic effect and/or elicits a desired biological and/or pharmacological effect.

The ammocidin compounds (e.g., ammocidin A, ammocidin B, ammocidin C, ammocidin D, ammocidin E, especially ammocidin A) and compound of Formula (1) (and sub-formulae thereof) may be effectively used for the treatment of proliferative disorders, including cancer and similar diseases. The compounds may be used to treat cancers characterized by one or more solid tumors. In other embodiments, the proliferative disorder is a blood cancer. In some embodiments, the proliferative disorder is myelodysplastic syndrome. In some embodiments, the proliferative disorder is leukemia.

In a preferred embodiment, naturally occurring ammocidin (e.g., ammocidin A) or a compound of Formula (1), (2a), (2b), (2c), (3a), (3b), or (3c) may be used to treat proliferative disorder such as acute myeloid leukemia, chronic myeloid leukemia, acute lymphoblastic leukemia, multiple myeloma, mixed lineage leukemia, brain tumor, glioblastoma, or lymphoma. Preferably the agent used is ammocidin A or a compound of Formula (3c).

The compounds disclosed herein may be used to treat acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary—cervical cancer, cholangiocarcinoma, chordoma, chronic lymphocytic leukemia (CLL), chronic myelogenous leukemia (CIL), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney-langerhans cell histiocytosis, laryngeal cancer, laryngeal cancer and papillomatosis, leukemia, lip and oral cavity cancer, liver cancer (primary), lung cancer, lung cancer, lymphoma-macroglobulinemia, Waldenström-Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenström Macroglobulinemia, Wilms Tumor.

In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonal antibodies. As such, the compounds disclosed herein may be administered as part of a combination treatment regime, for instance prior to or following surgery or prior to or following radiation treatment.

In some embodiments, the compounds disclosed herein may be administered in combination with one or more anticancer agents for example mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, epigenetic inhibitors, biological response modifiers, anti-metabolites, glycolysis inhibitors, glutamine metabolism inhibitors, anti-hormones, angiogenesis inhibitors, cytostatic agents anti-androgens, targeted antibodies, HMG-CoA reductase inhibitors, and prenyl-protein transferase inhibitors.

Exemplary anti-cancer agents include nucleoside analogues, antifolates, antimetabolites, topoisomerase I inhibitor, anthracyclines, podophyllotoxins, taxanes, vinca alkaloids, alkylating agents, platinum compounds, proteasome inhibitors, nitrogen mustards & oestrogen analogue, monoclonal antibodies, tyrosine kinase inhibitors, mTOR inhibitors, retinoids, immunomodulatory agents, histone deacetylase inhibitors, DNA methyl-transferase inhibitors, BCL-2 family protein inhibitors and combinations thereof.

In some embodiments, the additional anticancer agent can be one or more nucleoside analogs, for instance one or more azanucleosides.

In certain embodiments, the anti-cancer agent is selected from one or more of abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, recombinant hpv quadrivalent vaccine, recombinant hpv nonavalent vaccine, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, and pharmaceutically acceptable salts thereof.

The additional agents may be combined with the disclosed compounds in a single pharmaceutical formula (as defined herein) or may be administered separately.

In a preferred embodiment, naturally occurring ammocidin (e.g., ammocidin A) or a compound of Formula (1), (2a), (2b), (2c), (3a), (3b), or (3c) may be combined with one or more agents for the treatment of leukemia, for instance acute myeloid leukemia, chronic myeloid leukemia, acute lymphoblastic leukemia, or mixed lineage leukemia. Exemplary agents include vincristine, azacytidine, decitabine, cytarabine, daunomycin, venetoclax, ibrutinib, idelalisib, doxorubicin, idarubicin (or another anthracycline), L-asparaginase, PEG-L-asparaginase, cyclophosphamide, nelarabine, cladribine, fludarabine, mitoxantrone, etoposide, hydroxyurea, methotrexate, 6-mercaptopurine, azacytidine, decitabine, prednisone, dexamethasone, or other corticosteroid. Preferably the one or more agents for leukemia is combined with either ammocidin A or a compound of Formula (3c).

In some preferred embodiments, leukemia may be treated using a naturally occurring ammocidin (e.g., ammocidin A) or a compound of Formula (1), (2a), (2b), (2c), (3a), (3b), or (3c), in combination with venetoclax, one or more nucleoside analogs or azanucleosides (e.g., azacytidine, decitabine, cedazuridine) or both and venetoclax and one or more nucleoside analogs or azanucleosides. Preferably azacytidine, venetoclax, or both azacytidine and venetoclax, is combined with either ammocidin A or a compound of Formula (3c). In other embodiments, ammocidin A or a compound of Formula (3c) is administered in combination of decitabine and cedazuridine.

In some embodiments, the compounds disclosed herein may be administered to patients in need thereof without corresponding increase in multi-drug resistance. In such embodiments, the disclosed compounds may be administered alone or in combination with one or more additional anti-cancer agents, as defined herein. In some embodiments, the disclosed compounds can be used to treat cancer, for instance leukemias like AML, in patients that have developed multidrug resistance.

Multidrug resistance may be identified using methods known in the art. In some embodiments, the patient can have multidrug resistance characterized by abnormal levels of P-glycoprotein, including elevated levels of P-glycoprotein (P-gp). In some instances, the patient has multidrug resistance characterized by upregulation of P-gp, downregulation of antiapoptotic protein B-cell lymphoma (Bcl-2), or both upregulation of P-gp and downregulation of Bcl-2. The patient can have multidrug resistance characterized by overexpression of multidrug resistance-related protein (MRP1) or by overexpression of ABCC1 gene. The patient can have multidrug resistance characterized by overexpression of lung resistance protein (LRP). The patient can have multidrug resistance characterized by overexpression of glutathione S-transferase (GST), include GSTu, GSTp, or GSTn. The patient can have multidrug resistance characterized by upregulation of protein kinase C (PKC), including PKCα, PKC^{e}, and PKC^{q}. The patient can have multidrug resistance characterized by mutation of FMS-like tyrosine kinase 3 (FLT3). The patient can have multidrug resistance characterized by expression of Wilms Tumor (WT1). The patient can have multidrug resistance characterized by RAS mutation, for instance KRAS mutation, HRAS mutation, or NRAS mutation. The patient can have multidrug resistance characterized by mutation of one or more of IDH1, TP53, ASXL1, DNMT3A, CEBPA, IDH2, PTPN11. The patient can have multidrug resistance characterized by differentiation state such as the FAB classification including primitive (MO) or monocytic (M5) differenation.

The pharmaceutical compositions described herein may be prepared by any method known or hereafter developed in the art of pharmaceutics. In general, such preparatory methods include the step of bringing the active ingredient into association with one or more excipients and/or one or more other accessory ingredients, and then, if necessary and/or desirable, shaping and/or packaging the product into a desired single- or multi-dose unit.

Dosage forms for topical and/or transdermal administration of the scaffolds may include ointments, pastes, creams, lotions, gels, powders, solutions, sprays, inhalants and/or patches. Generally, the active component is admixed under sterile conditions with a pharmaceutically acceptable excipient and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxy ethylene sorbitan [Tween 60], polyoxy ethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and perfuming agents. In certain embodiments for parenteral administration, conjugates can be mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U. S. P. and isotonic sodium chloride solution, etc. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the active ingredient then depends upon its rate of dissolution which, in turn, may depend upon crystal size and crystalline form. In some embodiments, delayed absorption of a parenterally administered active ingredient is accomplished by dissolving or suspending the drug in an oil vehicle.

Compositions for rectal or vaginal administration are typically suppositories which can be prepared by mixing the conjugates with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

The compounds disclosed herein are useful in the treatment of various conditions for which inhibition of ATP synthase is beneficial. In particular the compounds are useful in the treatment of hematologic cancers includes myeloid leukemia (both chronic and acute), lymphoid leukemia, including chronic lymphocytic leukemia, non-Hodgkin's lymphoma and mantle cell lymphoma, and myeloma. The compounds may also be used to treat glioblastoma, including ENO1 deficient glioblastoma, prostate cancer, and lung cancer.

### EXAMPLES

The following examples are for the purpose of illustration of the invention only and are not intended to limit the scope of the present invention in any manner whatsoever.

### Example 1: Ammocidin a Fermentation

Ammocidin A was obtained by cultivation of Saccharothrix sp. AJ9571 provided by Ajinomoto Co., Inc (Kawasaki, Japan). The organism was plated on Bennett's agar (0.1% yeast extract, 0.1% beef extract, 0.2% N-Z Amine Type A, 1.0% dextrose, 2.0% agar, pH 7.0) and incubated at 30° C. for 3-7 days until sporulation. The seed culture was initiated using spores scraped from the solid culture into 250 mL Erlenmeyer flasks containing 50 mL of seed medium (1.0% soluble starch, 1.0% molasses (Plantation Blackstrap, Unsulfured), 1.0% peptone, 1.0% beef extract, pH 7.0), and incubated for 7 days at 30° C. while shaking at 220 RPM. Production cultures were carried out in multiple 250 mL Erlenmeyer flasks containing 50 mL of production media (2.0% glycerol, 1.0% molasses, 0.5% casamino acids, 0.10% peptone, 0.4% calcium carbonate, pH 7.2) and incubated at 30° C. for 7 days while shaking at 220 RPM.

After 7 days of fermentation, the mycelia were separated from the culture broth by centrifugation at 3000 g x 30 min. The culture broth was extracted 3× with 1 volume of ethyl acetate and the combined organic layers were washed with brine, dried with Na₂SO₄ and concentrated in vacuo. The crude extract was then subjected to chromatography with LH-20 resin using methanol as the mobile phase and the glycomacrolide containing fractions were identified by thin-layer-chromatography and pooled. The LH-20 fractions were then subjected to reverse phase HPLC using a Waters XBridge Prep C18 19×150 mm column with a 20-minute gradient from 70% A/30% B to 20% A/80% B, (Buffer A: 95% water, 5% acetonitrile, 10 mM ammonium acetate; Buffer B: 5% water, 95% acetonitrile, 10 mM ammonium acetate). Ammocidin A—RT 9.0 min. Fractions containing pure compound were lyophilized using a Genevac HT-6 to yield a white solid.

### Example 2: Ammocidin A Derivatization

### Derivatization with 3-(3-(but-3-yn-1-yl)-3H-diazirin-3-yl)propanoic acid

To a solution of 3-(3-(but-3-yn-1-yl)-3H-diazirin-3-yl)propanoic acid (4.5 mg, 0.027 mmol, Enamine, Kyiv, Ukraine) in dichloromethane (4.0 mL) on ice, was added bromo-tris-pyrrolidinophosphonium hexafluorophosphate (PyBrop, 13.3 mg, 0.28 mmol) and diisopropylethyl amine (DIPEA, 31 μL, 0.177 mmol). The resulting solution was stirred at 0° C. for 10 min. Ammocidin A (20 mg 0.018 mmol) was added, followed by a crystal of 4-dimethylaminopyridine (DMAP). The resulting solution was warmed to room temperature overnight (16 h). The reaction was monitored by TLC (90:10 CHCl₃:MeOH) and quenched with 100 μL of MeOH and then concentrated. The resulting residue was diluted in EtOAc (20 mL) and washed with 1 M HCl (5 mL). The aqueous layer was extracted twice with EtOAC (2×10 mL). The organic extracts were combined and washed with NaHCO₃ (5 mL) and brine (5 mL), dried with anhydrous sodium sulfate and concentrated in vacuo. The resulting residue was dissolved in 800 μL of MeOH and purified by reversed phase HPLC using a Waters XBridge Prep C18 19×150 mm column, with a 20-minute gradient from 32% to 77% acetonitrile in water, with 25 mM ammonium bicarbonate. The fractions containing the desired product (rt=16 min) were combined and lyophilized to afford the product as a white solid (0.6 mg, 2.5% isolated yield). HRMS (ESI-TOF MS) m/z 1287.6854 (M+Na)⁺ calculated 1287.6866, observed (0.9 ppm).

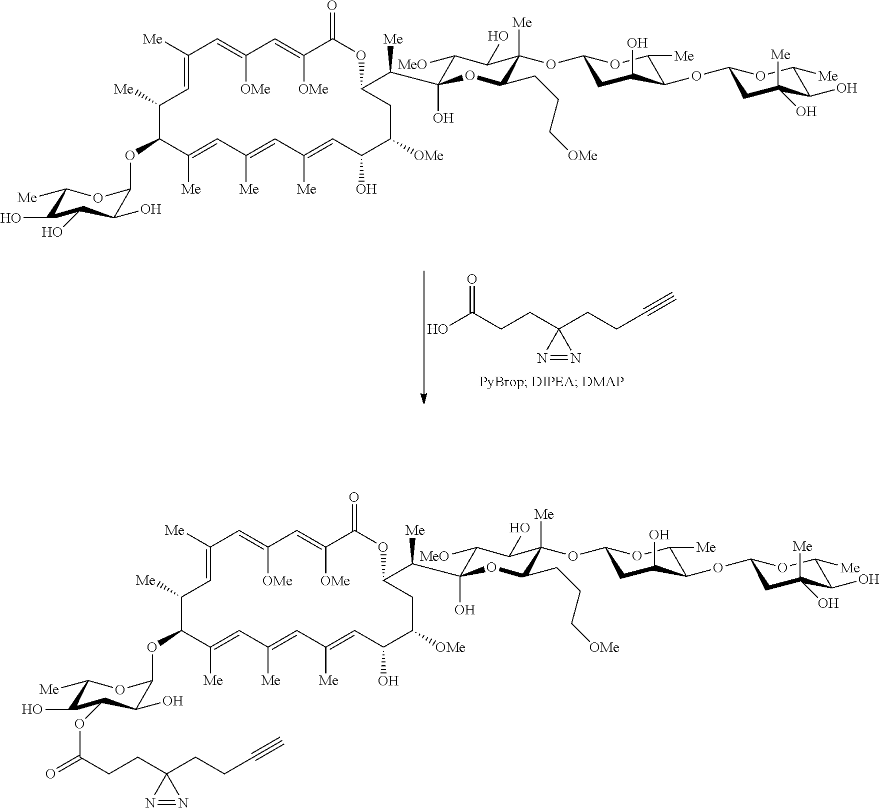

Regiochemistry of addition was confirmed by multidimensional NMR.

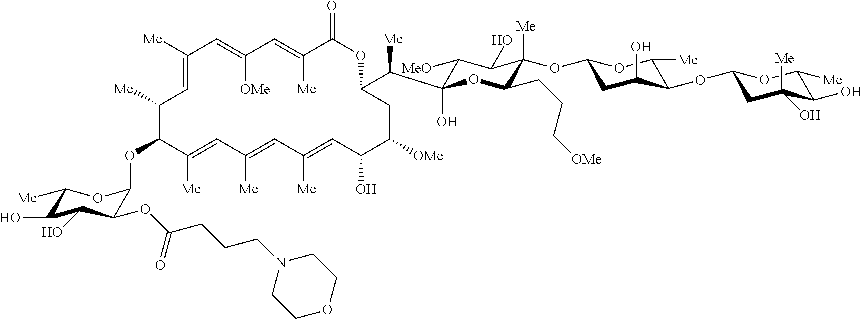

### Derivatization with 4-morpholinobutanoic acid

To a solution of 4-morpholinobutanoic acid (2.2 mg, 0.013 mmol) in dichloromethane (2.0 mL) on ice, was added bromo-tris-pyrrolidinophosphonium hexafluorophosphate (PyBrop, 7 mg, 0.14 mmol) and diisopropylethyl amine (DIPEA, 16 μL, 0.09 mmol). The resulting solution was stirred at 0° C. for 10 min. Ammocidin A (10 mg 0.009 mmol) was added, followed by a crystal of 4-dimethylaminopyridine (DMAP). The resulting solution was warmed to room temperature overnight (16 h). The reaction was monitored by TLC (90:10 CHCl₃:MeOH) and quenched with 100 μL of MeOH and then concentrated. The resulting residue was diluted in EtOAc (20 mL) and washed with 1 M HCl (5 mL). The aqueous layer was extracted twice with EtOAC (2×10 mL). The organic extracts were combined and washed with NaHCO₃ (5 mL) and brine (5 mL), dried with anhydrous sodium sulfate and concentrated in vacuo. The resulting residue was dissolved in 800 μL of MeOH and purified by reversed phase HPLC using a Waters XBridge Prep C18 19×150 mm column, with a 20-minute gradient from 32% to 77% acetonitrile in water, with 25 mM ammonium bicarbonate. Two major products were noted as distinct peaks and were collected in separate fractions. HRMS (ESI-TOF MS) confirmed addition of the ester (M+H)⁺ 1312.7412 m/z calculated, 1312.7400 m/z observed (0.9 ppm). 2D NMR (HSQC, HMABC), confirmed the two products as a the 2′ and 3′ esters.

The 3-(4-methylpiperazin-1-yl) butanoic ester was prepared using similar methods and the addition was confirmed by HRMS (ESI-TOF MS) (M+H)⁺ 1325.7729 m/z calculated, 1325.7704 observed (1.9 ppm).

### Example 3: Apoptolidin Biosynthesis Modification

The translated sequences of the five gene (R)-2-methoxymalonyl-acyl carrier protein (MeOM-ACP) contiguous gene cassette (apoK-M2) has broad sequence identity to fkbG-K which encode the biosynthesis of this extender unit from the FK520 gene cluster in Streptomyces hygroscopicus, with ApoJ displaying 53% identity to the acyl carrier protein for the extender unit. However, while MeOMal-ACP has been reported to function as an extender unit by intercepting polyketide synthase in trans, it never been reported as chain initiator.

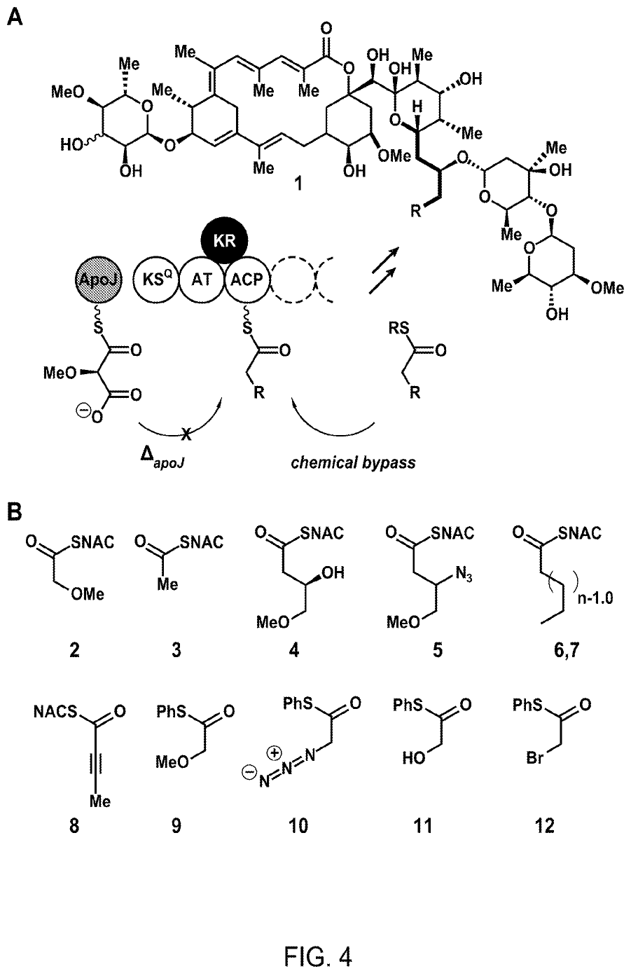

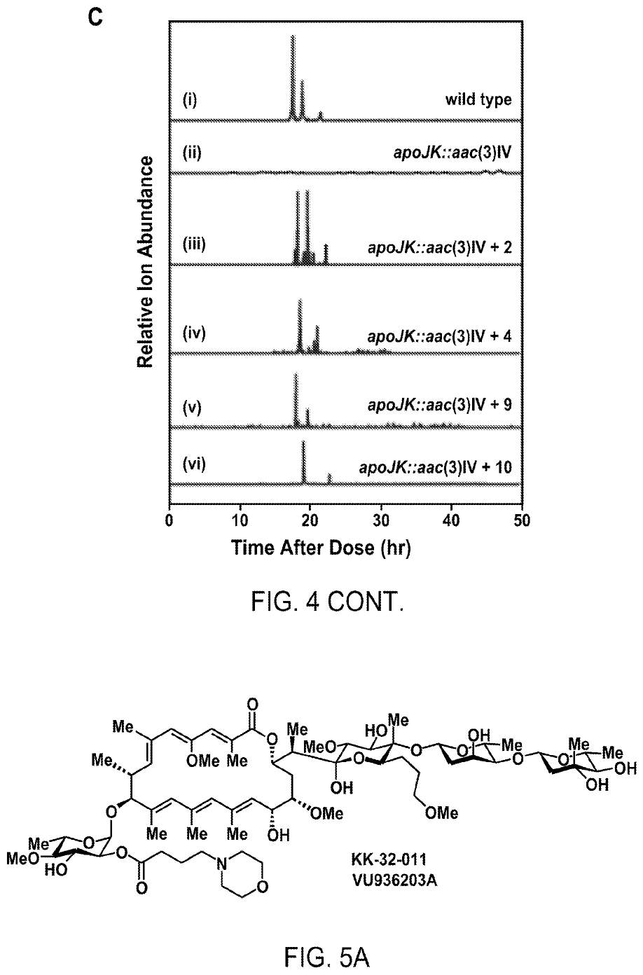

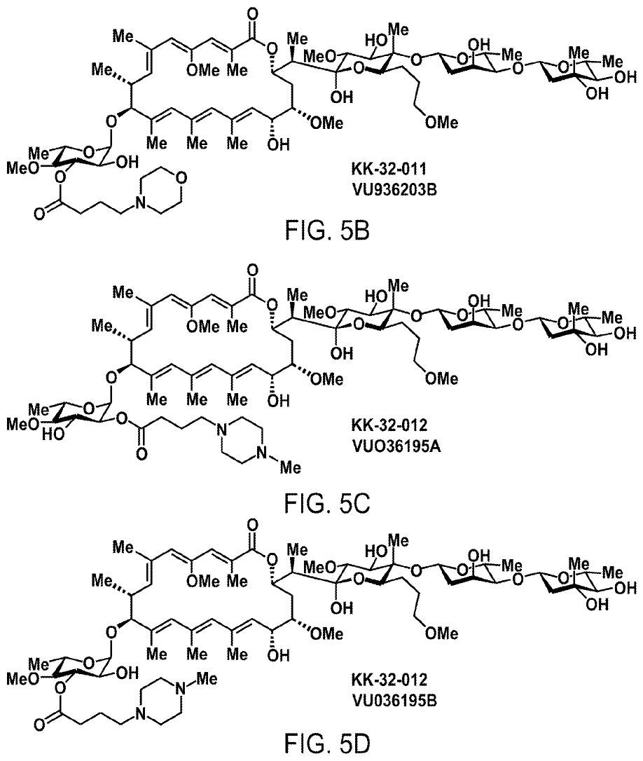

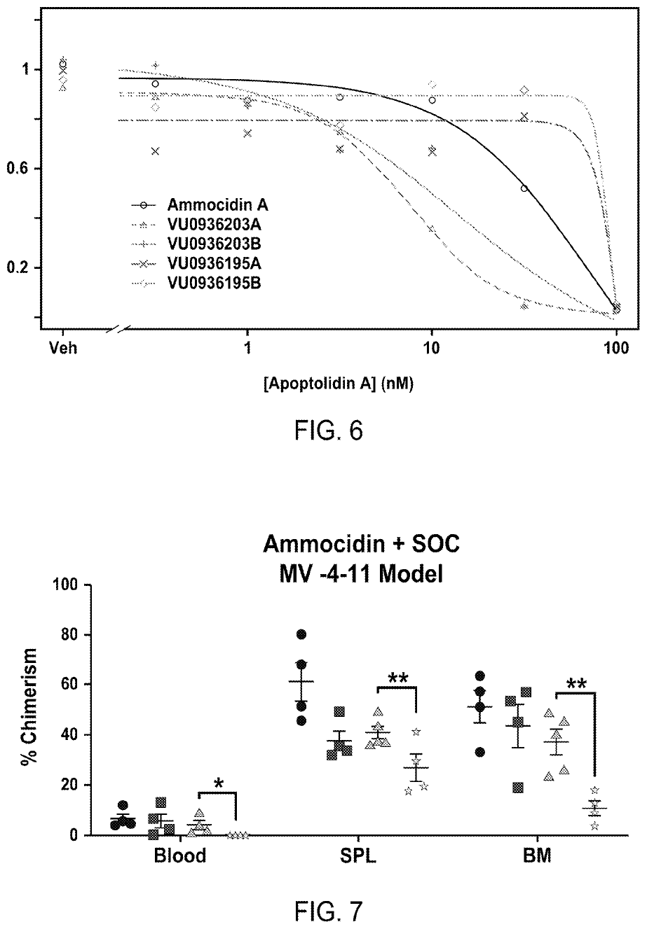

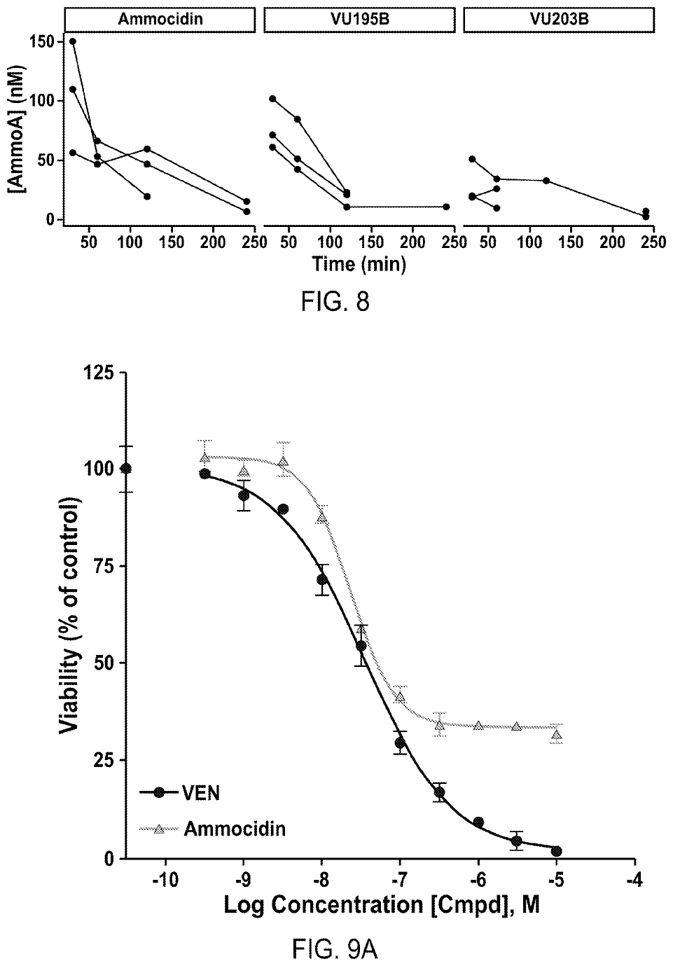

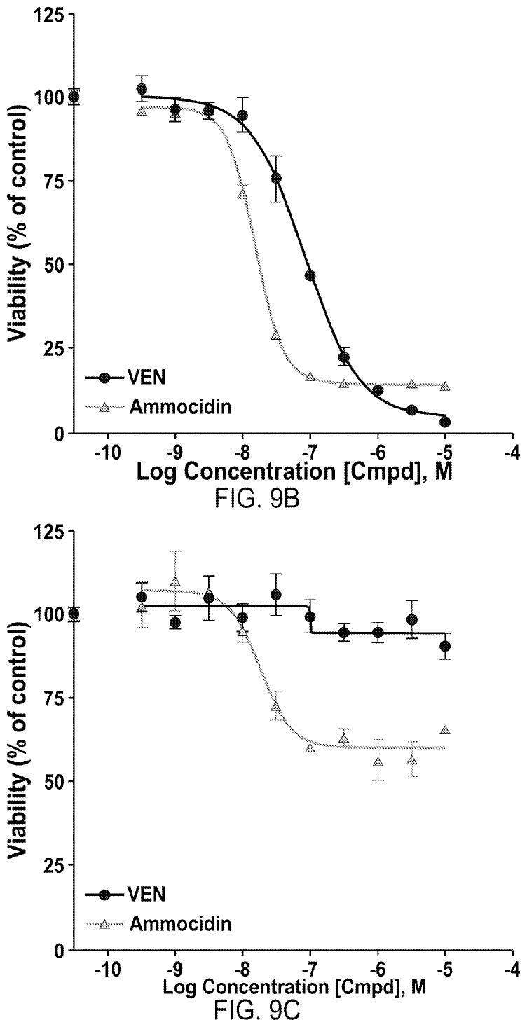

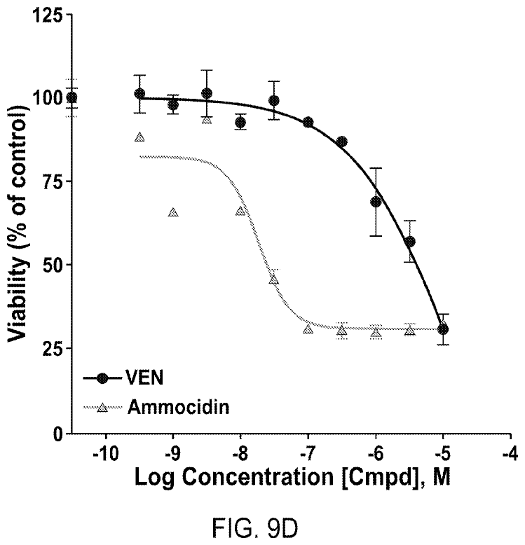

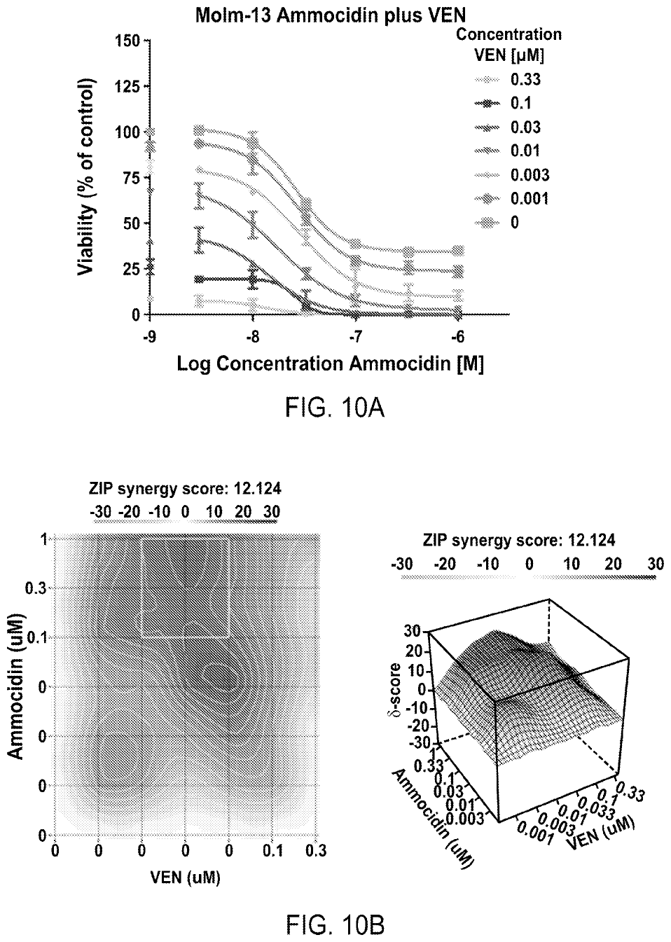

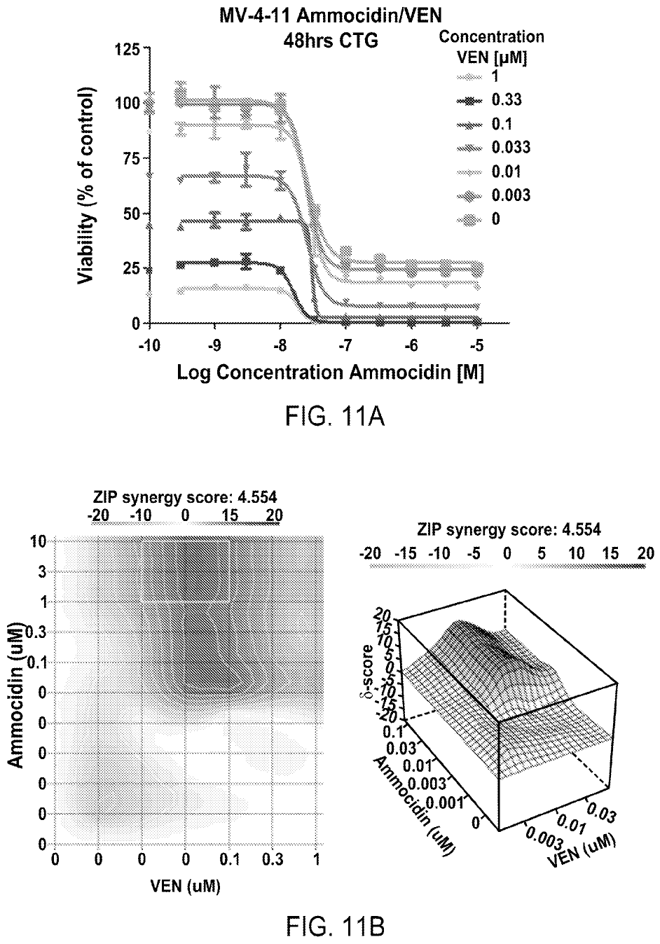

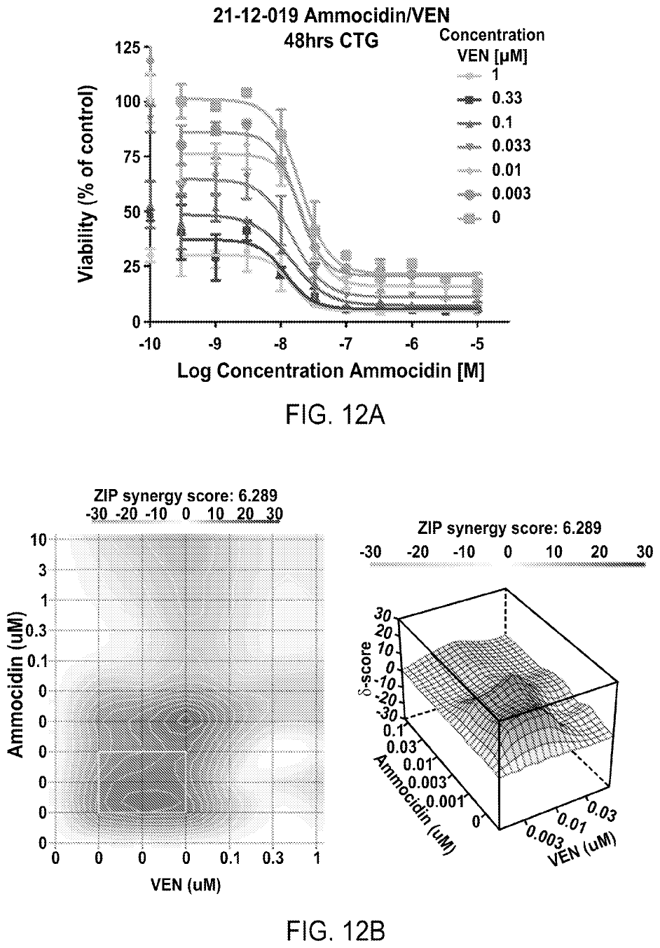

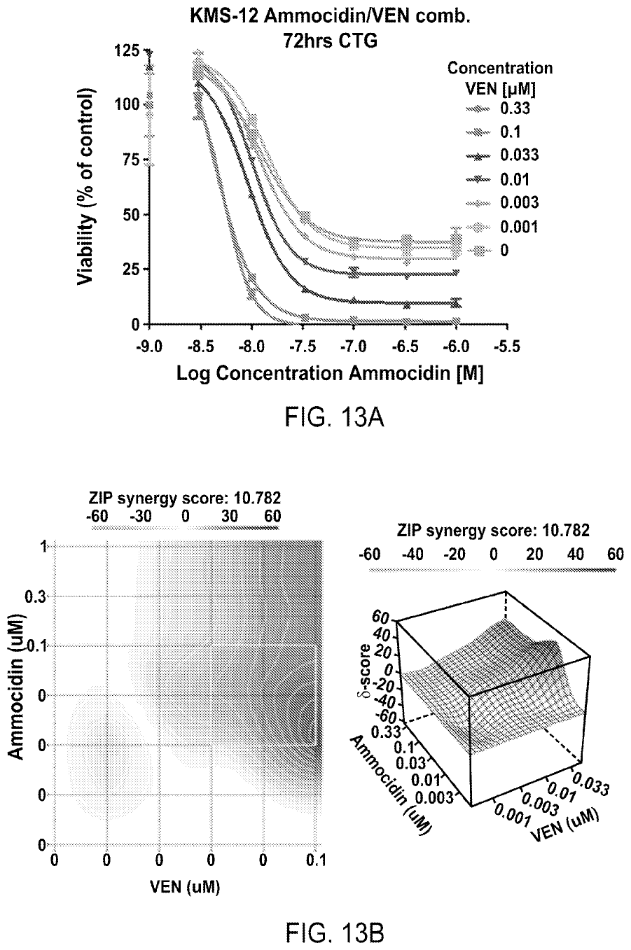

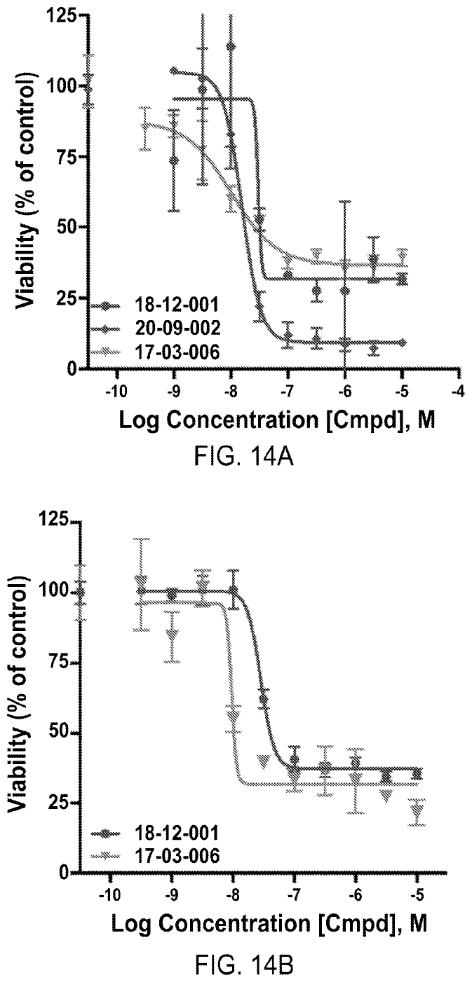

To disrupt apoJ in this cluster, we employed two step PCR-targeting replacement, in which genes were first replaced by antibiotic resistance markers in fosmids containing the apo gene cluster and subsequently transferred into Nocardiopsis to select for double crossover events. Attempts to replace 291 bp apoJ did not yield recombinant clones, however double gene replacement of apoJK was successful, resulting in a mutant strain Nocardiopsis sp. FU40 apoJK::aac(3)IV. The translated apoK gene possesses 65% identity with FkbK, an oxidase responsible for 3-OH dehydrogenation of glyceryl-ACP en route to hydroxymalonate, and its deletion is predicted to have no effect of downstream apoptolidin biosynthesis. LC-MS analysis of extracts of fermentation cultures of this strain demonstrated a complete abolishment of production of all apoptolidins, supporting the hypothesis of (R)-2-methoxymalonyl-ACP biosynthetic initiation (FIG. 4).

Synthetic thioesters of chain initiating and extension building blocks and intermediates have been shown to load KS active site cysteine thiols domains in vitro, and have also been successfully employed in chemical complementation studies of blocked polyketide biosynthetic pathways. The chemical rescue of the apoJK knockout strain was performed with the N-acetylcysteamine, (NAC) thioester of (R)-2-methoxymalonate. Initial studies of synthesized MeOMe-SNAC added to early stage Nocardiopsis growth cultures substantially restored apoptolidin A biosynthesis. Optimal incorporation efficiency was determined by evaluating pulsed dosing schedules in which 60 ug/mL were fed in equal portions over the seven day fermentation. It was determined that pulsed supplementation of MeOMe-SNAC with 50 μL aliquots of 8 mg/mL DMSO starting on the 2nd day of seed culture yielded the best results with production of apoptolidin A restored to near wildtype levels. These results are consistent with the site of acylation of the first polyketide synthase protein ApoS1 being the active cite cysteine in the second KS domain.

### Example 4: Bypass Fermentation with Synthetic Starter Units

Thiophenyl esters of methoxyacetic acid 9, 2-azidoacetic acid 10, 2-hydroxyacetic acid 11. and 2-bromoacetic acid 12 were prepared and supplemented into Nocardiopsis sp. FU40 apoJK::aac(3)IV cultures. LC/MS analysis of extracts revealed that the phenylthio ester of methoxyacetate successfully complemented the apoJK deletion (FIG. 4C, trace v) and that 2-azidoacetic acid bypass resulted in a new metabolite of m/z=1139 with a UV max of 290 and 330 nm consistent with the properties of other apoptolidins suggesting successful incorporation of the azide at C28 (FIG. 4C, trace vi). To confirm the identity of this newly observed compound, we performed collision induced dissociation studies on the putative azide containing analog. The three sugars of the apoptolidins provide diagnostic fragments for identification. Loss of the C27 sugar with dehydration yielded fragments with m/z 306 and 835. Subsequent loss of the C9 sugar resulted in fragments with m/z of 163 and 675. Finally observation of fragmentation across the C22-C23 bond and dehydration (m/z of 457) confirmed incorporation of the azide at the terminal end of apoptolidin.

### Example 5: Cell Proliferation Assay (CellTitre-Glo)

Compounds were diluted in DMSO (<0.05% DMSO) and dispensed into a 384-well plate using the Echo 555 liquid handler (Labcyte). Following the addition of compounds, cells were pipetted into the 384-well plates at a concentration of between 2,000 and 8,000 cells per well in IMDM or RPMI media, as noted above, supplemented with 10% FBS and incubated at 37° C., 5% CO2 in a tissue culture incubator. Plates were incubated for 48 hours, and cell viability was measured using the CellTiter-Glo reagent (Promega). Percent viability was defined as relative luminescence units (RLU) of each well divided by the RLU of cells in DMSO control. Dose-response curves and GI50 values were determined using linear regression of double-log transformed data (GraphPad Prism version 6.0 h). Control bone marrow-derived CD34+ cells were purchased from STEMCELL Technologies.

100 μl of suspension cells at 100,000 cells ml-1 were added to wells of a microtiter plate precoated with 0.5 μl of test compound at 200× in dimethylsulfoxide and incubated for 48-72 h. MTT reagent was dissolved in fresh media at 1 mg ml-1 and 100 μl was added to each well to achieve a final concentration of 0.5 mg ml-1 and incubated for 2 h at 37° C. Cells were centrifuged at 800 g for 5 min and decanted. MTT crystals were redissolved in 100 μl of dimethylsulfoxide, allowed to incubate for 5 min at room temperature, and read at 560 nm using a SpectraMax plus 384 plate reader (Molecular Devices). Absorbance values were normalized by background subtraction (wells without cells) such that vehicle-treated cells had a viability of 1.0. Concentration-response curves were fit using the DRC R package with a four-parameter log-logistic function. Statistical testing of differences between concentration-response curves was carried out using the EDcomp function with default parameters for comparison of half maximal inhibitory concentration values and the paramcomp function for comparison of other parameters. The compounds listed in the table below were prepared using techniques described herein:

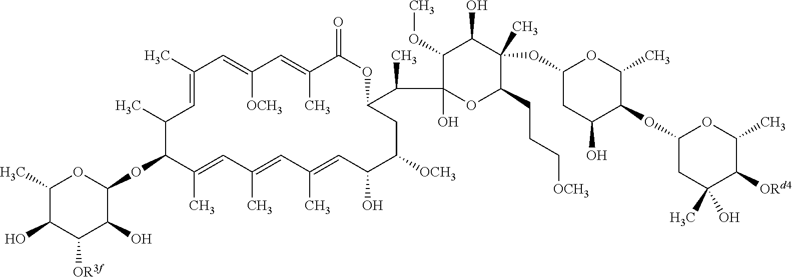

| R^{3f} | R^{d4} | MV-4-11 Cytotoxicity IC₅₀ |
|---|---|---|
| 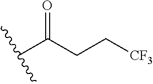 [SMILES: *C(=O)CCC(F)(F)F] | | 3.9 nM |
| 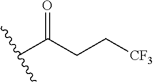 [SMILES: *C(=O)CCC(F)(F)F] | 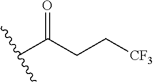 [SMILES: *C(=O)CCC(F)(F)F] | 15 nM |
| 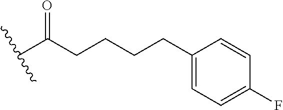 [SMILES: *C(=O)CCCCc1ccc(F)cc1] | | 1.8 nM |
| 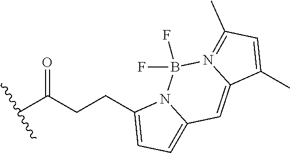 | | 15 nM |
| 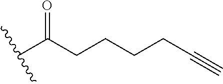 [SMILES: *C(=O)CCCCC#C] | | 1.8 nM |

### Example 6: Pharmacokinetic Studies

All animal experiments were conducted in accordance with guidelines approved by the IACUC at Vanderbilt University Medical Center. Pharmacokinetics of ammocidin in NSGS male mice in biological triplicate were assessed in whole blood after dosing with ammocidin alone 0.5 mg kg−1 intraperitoneal. Whole-blood samples were collected up to in EDTA tubes for analysis of plasma. Blood plasma was mixed 1:1 with an internal standard solution consisting of 1 μM Apop A in PBS. Metabolites were extracted with 200 μl of ethyl acetate, evaporated to dryness and resuspended in 50 μl of MeOH. Ammo A concentration was determined using liquid chromatography-MS (Thermo TSQ Quantum Access Max) with technical duplicates on a 50×1.8 mm C18 column, isocratic 60/40 H2O/acetonitrile+10 mM ammonium acetate at 250 μl min−1 in electrospray ionization-positive mode monitoring Ammo A (1139.7→208.8, collision energy (CE) 19 V, retention time (RT)=1.00 min) and Apop A (1146.68→805.46, CE 19 V, RT=1.66 min).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound having the formula: 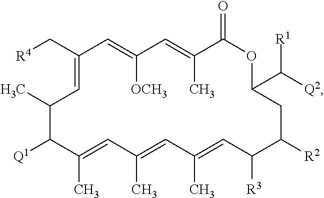
or a pharmaceutically acceptable salt thereof,
wherein
R¹ is selected from H, OH, C₁₋₈alkyl.
R² is selected from H, OH, C₁₋₈alkyl.
R³ is selected from H, OH, C₁₋₈alkyl.
R⁴ is selected from H, OH, C₁₋₈alkyl.
Q¹ is a group having the formula: 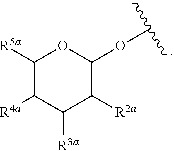
wherein
R^{2a} is selected from —R^{2a}*, —OR^{2a}*, OP(O)(OR^{2a}*)₂, OP(O)(OR^{2a}*)(N(R^{2a}*)₂, —N(R^{2a}*)₂, —N(R^{2a}*)₃, —C(O)R^{2a}*, —C(O)OR^{2a}*, —OC(O)R^{2a}*, —OC(O)OR^{2a}*, —NR^{2a}*C(O)R^{2a}*, —C(O)N(R^{2a}*)₂, NR^{2a}*C(O)OR^{2a}*, —OC(O)N(R^{2a}*)₂, —NR^{2a}*C(O)N(R^{2a}*)₂; —Cl, —F, —Br, —I, —NO₂, —CN, —N₃, —(OCH₂CH₂)ₘ—OH;
R^{2a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{2a}* may be substituted one or more times by —OH, —COOH, —, —PPh₃, —CH₂NEt₃, —CH₂NMe₃, —NHC(═NH)NH₂, a rhodamine dye, a bisquinolinium, —NH₂, —Cl, —F, —Br, —I, —NO₂, —CN, —N₃, PO(OH)₂, —(OCH₂CH₂)ₘ—OH; C₁₋₈heterocycyl, aryl, —OC₁₋₈heterocycyl, or C₁₋₈alkoxy,
wherein any two or more of R^{2a}* may together form a ring;
R^{3a} is selected from —R^{3a}*, —OR^{3a}*, OP(O)(OR^{3a}*)₂, OP(O)(OR^{3a}*)(N(R^{3a}*)₂, —N(R^{3a}*)₂, —N(R^{3a}*)₃—C(O)R^{3a}*, —C(O)OR^{3a}*, —OC(O)R^{3a}*, —OC(O)OR^{3a}*, —NR^{3a}*C(O)R^{3a}*, —C(O)N(R^{3a}*)₂, NR^{3a}*C(O)OR^{3a}*, —OC(O)N(R^{3a}*)₂, —NR^{3a}*C(O)N(R^{3a}*)₂; —Cl, —F, —Br, —I, —NO₂, —CN, —N₃, —(OCH₂CH₂)ₘ—OH;
R^{3a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{3a}* may be substituted one or more times by —OH, —COOH, —PPh₃, —CH₂NEt₃, —CH₂NMe₃, —NHC(═NH)NH₂, a rhodamine dye, a bisquinolinium, —NH₂, —Cl, —F, —Br, —I, —NO₂, —CN, —N₃, PO(OH)₂, —(OCH₂CH₂)ₘ—OH; C₁₋₈heterocycyl, aryl, —OC₁₋₈heterocycyl, or C₁₋₈alkoxy,
wherein any two or more of R^{3a}* may together form a ring;
R^{4a} is selected from —R^{4a}*, —OR^{4a}*, OP(O)(OR^{4a}*)₂, OP(O)(OR^{4a}*)(N(R^{4a}*)₂, —N(R^{4a}*)₂, —N(R^{4a}*)₃, —C(O)R^{4a}*, —C(O)OR^{4a}*, —OC(O)R^{4a}*, —OC(O)OR^{4a}*, —NR^{4a}*C(O)R^{4a}*, —C(O)N(R^{4a}*)₂, NR^{4a}*C(O)OR^{4a}*, —OC(O)N(R^{4a}*)₂, —NR^{4a}*C(O)N(R^{4a}*)₂; —Cl, —F, —Br, —I, —NO₂, —CN, —N₃, —(OCH₂CH₂)ₘ—OH;
R^{4a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{4a}* may be substituted one or more times by —OH, —COOH, —PPh₃, —CH₂NEt₃, —CH₂NMe₃, —NHC(═NH)NH₂, a rhodamine dye, a bisquinolinium, —NH₂, —Cl, —F, —Br, —I, —NO₂, —CN, —N₃, PO(OH)₂, —(OCH₂CH₂)ₘ—OH; C₁₋₈heterocycyl, aryl, —OC₁₋₈heterocycyl, or C₁₋₈alkoxy;
wherein any two or more of R^{4a}* may together form a ring;
R^{5a} is selected from —R^{5a}*, —OR^{5a}*, OP(O)(OR^{5a}*)₂, OP(O)(OR^{5a}*)(N(R^{5a}*)₂, —N(R^{5a}*)₂, —N(R^{5a}*)₃, —C(O)R^{5a}*, —C(O)OR^{5a}*, —OC(O)R^{5a}*, —OC(O)OR^{5a}*, —NR^{5a}*C(O)R^{5a}*, —C(O)N(R^{5a}*)₂, NR^{5a}*C(O)OR^{5a}*, —OC(O)N(R^{5a}*)₂, —NR^{5a}*C(O)N(R^{5a}*)₂; —Cl, —F, —Br, —I, —NO₂, —CN, —N₃, —(OCH₂CH₂)ₘ—OH;
R^{5a}* is in each case independently selected from H, C₁₋₁₀alkyl, C₂₋₁₀alkenyl, C₂₋₁₀alkynyl, aryl, C₃₋₁₀cyclolkyl, C₃₋₁₀cycloalkenyl, C₇₋₁₀cycloalkynyl, aryl, C₁₋₁₀heterocycyl, C₁₋₁₀heteroaryl; wherein each R^{5a}* may be substituted one or more times by —OH, —COOH, —PPh₃, —CH₂NEt₃, —CH₂NMe₃, —NHC(═NH)NH₂, a rhodamine dye, a bisquinolinium, —NH₂, —Cl, —F, —Br, —I, —NO₂, —CN, —N₃, PO(OH)₂, —(OCH₂CH₂)ₘ—OH; C₁₋₈heterocycyl, aryl, —OC₁₋₈heterocycyl, or C₁₋₈alkoxy;
wherein R^{p} is in each case selected from H, C₁₋₁₀alkyl, and aryl;
wherein any two or more of R^{5a}* may together form a ring;
wherein any two of more of R^{2a}, R^{3a}, R^{4a}, and R^{5a} may together form a ring;
Q² is a group having the formula: 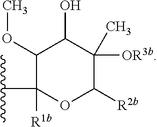
wherein:
R^{1b} is selected from H, OH, C₁₋₈alkyl, and OC₁₋₈alkyl;
R^{2b} is selected from —R^{2b}*, —OR^{2b}*, OP(O)(OR^{2b}*)₂, OP(O)(OR^{2b}*)(N(R^{2b}*)₂, —N(R^{2b}*)₂, —N(R^{2b}*)₃, —C(O)R^{2b}*, —C(O)OR^{2b}*, —OC(O)R^{2b}*, —OC(O)OR^{2b}*, —NR^{2b}*C(O)R^{2b}*, —C(O)N(R^{2b}*)₂, NR^{2b}*C(O)OR^{2b}*, —OC(O)N(R^{2b}*)₂, —NR^{2b}*C(O)N(R^{2b}*)₂; —Cl, —F, —Br, —I, —NO₂, —CN, —N₃, —(OCH₂CH₂)ₘ—OH;
R^{2b}* is in each case independently selected from H, C₁₋₈alkyl, C₁₋₈alkenyl, C₁₋₁₀alkynyl, aryl, C₃₋₈cyclolkyl, C₃₋₈cycloalkenyl, C₇₋₁₀cycloalkynyl, aryl, C₁₋₈heterocycyl, C₃₋₈heteroaryl; wherein each R^{2b}* may be substituted one or more times by —OH, —COOH, —PPh₃, —CH₂NEt₃, —CH₂NMe₃, —NHC(═NH)NH₂, a rhodamine dye, a bisquinolinium, —NH₂, —Cl, —F, —Br, —I, —NO₂, —CN, —N₃, PO(OH)₂, —(OCH₂CH₂)ₘ—OH; C₁₋₈heterocycyl, aryl, —OC₁₋₈heterocycyl, or C₁₋₈alkoxy,
wherein any two or more of R^{2b}* may together form a ring;
R^{3b} is selected from H or a group having the formula: 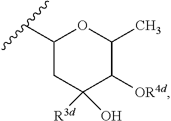
wherein
R^{3d} is selected from H and CH₃; and
R^{4d} is selected from H and a group having the formula: 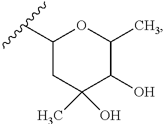
wherein m is in each case selected from 1-100;
with the proviso that when all of R^{2a}, R^{3a}, and R^{4a} are OH, R^{2b} is not CH₂CH₂CH₂OCH₃.

2. The compound according to claim 1, wherein the compound is a compound of Formula (2a), (2b), or (2c):

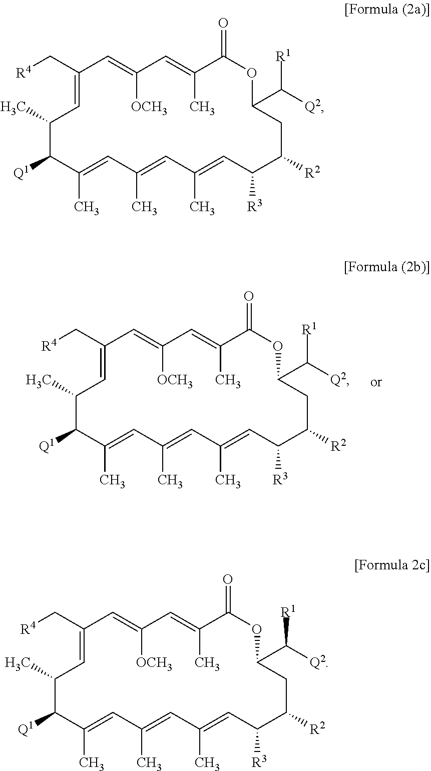

3. The compound according to claim 1, wherein Q¹ is an (L) sugar having the conformation:

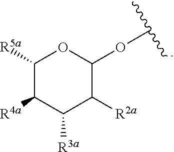

4. The compound according to claim 1, wherein Q¹ has the structure:

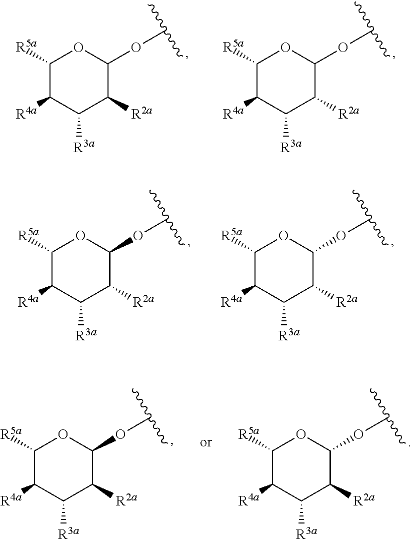

5. The compound according to claim 1, wherein Q² has the formula:

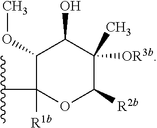

6. (canceled)

7. The compound according to claim 1, wherein R^{3b} has the formula:

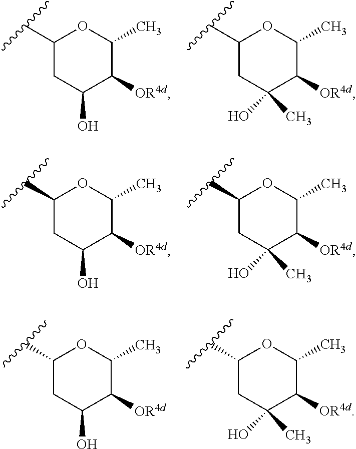

8. The compound according to claim 1, wherein R^{4d} has the formula:

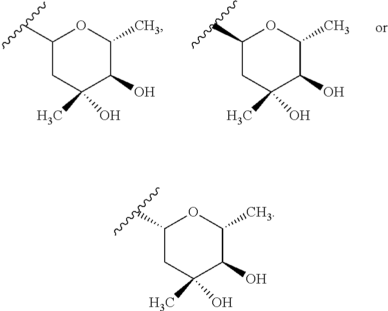 [SMILES: *C1C[C@@](C)(O)[C@H](O)[C@@H](C)O1 ; *[C@@H]1C[C@@](C)(O)[C@H](O)[C@@H](C)O1 ; *[C@H]1C[C@@](C)(O)[C@H](O)[C@@H](C)O1]

9. The compound according to claim 2, wherein R^{5a} is selected from CH₃ and CH₂OH.

10. The compound according to claim 2, wherein R^{4a} and R^{2a} are each OH.

11. The compound according to claim 2, wherein R^{3a} and R^{2a} are each OH.

12. The compound according to claim 2, wherein R^{3a} and R^{4a} are each OH.

13-44. (canceled)

45. The compound according to claim 1, where the compound has the Formula (3a), (3b), or (3c): 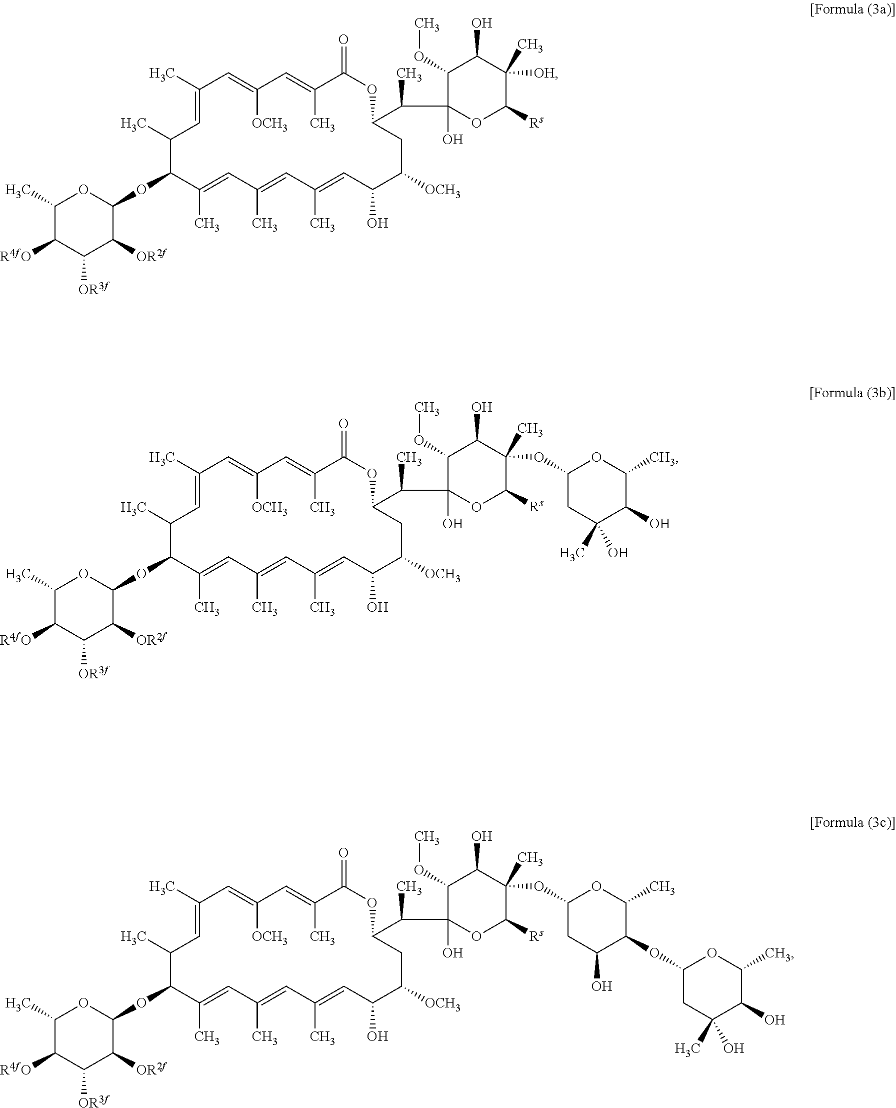
or a pharmaceutically acceptable salt thereof,
wherein R^{2f} is —R^{f}*, —C(O)R^{f}*, —C(O)OR^{f}*, —OC(O)R^{2b}*, —OC(O)OR^{f}*, or C(O)N(R^{f}*)₂;
wherein R^{3f} is —R^{f}* —C(O)R^{f}*, —C(O)OR^{f}*, —OC(O)R^{2b}*, —OC(O)OR^{f}*, or C(O)N(R^{f}*)₂;
wherein R^{4f} is —R^{f}*, —C(O)R^{f}*, —C(O)OR^{f}*, —OC(O)R^{2b}*, —OC(O)OR^{f}*, or C(O)N(R*)₂;
R^{f}* is in each case independently selected from H, substituted or unsubstituted C₁₋₈alkyl, substituted or unsubstituted C₁₋₈alkenyl, substituted or unsubstituted C₁₋₁₀alkynyl, substituted or unsubstituted aryl, substituted or unsubstituted C₃₋₈cyclolkyl, substituted or unsubstituted C₃₋₈cycloalkenyl, substituted or unsubstituted C₇₋₁₀cycloalkynyl, aryl, substituted or unsubstituted C₁₋₈heterocycyl, substituted or unsubstituted C₃₋₈heteroaryl;
R^{s} is selected from H, substituted or unsubstituted C₁₋₈alkyl, substituted or unsubstituted C₁₋₈alkenyl, substituted or unsubstituted C₁₋₁₀alkynyl, substituted or unsubstituted aryl, substituted or unsubstituted C₃₋₈cyclolkyl, substituted or unsubstituted C₃₋₈cycloalkenyl, substituted or unsubstituted C₇₋₁₀cycloalkynyl, substituted or unsubstituted aryl, C₁₋₈heterocycyl, substituted or unsubstituted C₃₋₈heteroaryl;
provided that R^{2f}, R^{3f} and R^{4f} are not simultaneously H when R^{s} is CH₂CH₂CH₂OCH₃.

46. The compound according to claim 45, wherein R^{2f} is —C(O)R^{2f}*, and R^{3f} and R^{4f} are each H and R^{s} is CH₂CH₂CH₂OCH₃.

47. The compound according to claim 45, wherein R^{3H} is —C(O)R^{f}*, and R^{2f} and R^{4f} are each H and R^{s} is CH₂CH₂CH₂OCH₃.

48. The compound according to claim 45, wherein R^{4f} is —C(O)R^{f}, and R^{2f} and R^{3f} are each H and R^{s} is CH₂CH₂CH₂OCH₃.

49-50. (canceled)

51. A method of treating cancer in a patient in need thereof, comprising administering the compound according to claim 1 to the patient.

52-53. (canceled)

54. The method of claim 51, wherein the cancer comprises chronic myeloid leukemia, acute myeloid leukemia, lymphoid leukemia, chronic lymphocytic leukemia, non-Hodgkin's lymphoma, mantel cell lymphoma, or myeloma.

55-58. (canceled)

59. A method of treating leukemia in a patient in need thereof, comprising administering ammocidin to the patient.

60-61. (canceled)

62. The method of claim 59, wherein the cancer comprises chronic myeloid leukemia, acute myeloid leukemia, lymphoid leukemia, chronic lymphocytic leukemia, non-Hodgkin's lymphoma, mantel cell lymphoma, or myeloma.

63-133. (canceled)
